(19) United States
(12) Patent Application Publication
(10) Pub. No.: US 2026/0250273 A1
(43) Pub. Date: Aug. 27, 2026

(54) CRYSTAL FORM OF COMPOUND, AND PHARMACEUTICAL COMPOSITION AND USE THEREOF

(71) Applicant: SHENZHEN KEYE LIFE TECHNOLOGIES CO., LTD, Shenzhen (CN)

(72) Inventors: Yandong ZHANG, Shenzhen (CN); Xianglu LI, Shenzhen (CN)

(73) Assignee: SHENZHEN KEYE LIFE TECHNOLOGIES CO., LTD, Shenzhen (CN)

(21) Appl. No.: 19/161,731

(22) PCT Filed: Nov. 30, 2023
(86) PCT No.: PCT/CN2023/135583

§ 371 Date: Sep. 3, 2025

(30) Foreign Application Priority Data
May 15, 2023 (CN) 202310543446.8

(51) Int. Cl. C07D 409/14; A61K 31/4184

(52) U.S. Cl. CPC C07D 409/14; A61K 31/4184

(57) ABSTRACT

A crystal form of a compound, and a pharmaceutical composition and the use thereof are provided. Specifically, different crystal forms of 1-(3-cyano-5-methylthiophen-2-yl)-N-(6-methoxy-1H-benzo[d]imidazol-2-yl)-2,5-dimethyl-1H-pyrrole-3-carboxamide, a pharmaceutical composition including the crystal form and the use are provided. An X-ray powder diffraction (XRPD) pattern of the crystal form has characteristic diffraction peaks at the following 2θ angles: 5.36±0.2°, 12.17±0.2° and 10.85±0.2°.

### CROSS REFERENCE TO THE RELATED APPLICATIONS

This application is the national phase entry of International Application No. PCT/CN2023/135583, filed on Nov. 30, 2023, which is based upon and claims priority to Chinese Patent Application No. 202310543446.8, filed on May 15, 2023, the entire contents of which are incorporated herein by reference.

### TECHNICAL FIELD

The present invention belongs to the field of pharmaceutical chemistry, relates to different crystal forms of 1-(3-cyano-5-methylthiophen-2-yl)-N-(6-methoxy-1H-benzo[d]imidazol-2-yl)-2,5-dimethyl-1H-pyrrole-3-carboxamide (hereinafter referred to as “compound of Formula (1)”), and further relates to a pharmaceutical composition including the crystal form and the use.

### BACKGROUND

DHX33 belongs to a DEAD/H-box RNA helicase family. DEAD/H is an abbreviation for an amino acid sequence Asp-Glu-Ala-Asp/His. This motif, together with several other conserved amino acid motifs, are found in the primary protein sequences of RNA helicase family members, and is highly involved in nucleic acid substrate binding and adenosine triphosphate (ATP) hydrolysis. Although these family members share identical sequence motifs, each RNA helicase has its own specific substrate selectivity and unique biological functions. Human DHX33 protein has a molecular weight of 72 kDa and has the function of unwinding nucleic acids. Through bioenergy released from ATP hydrolysis, DHX33 protein drives conformational changes in RNA and protein complexes, thereby participating in a variety of RNA metabolic activities, including RNA transcription, splicing, editing, translation, and degradation. The function of DHX33 is not simply limited to the modification of RNA molecules. Studies have shown that, apart from unwinding RNA double-strands, DHX33 is also involved in DNA metabolism. Specifically, DHX33 protein can unwind a double-stranded structure of DNA, and plays an important role in a gene expression process.

Previous research indicates that DHX33 influences DNA methylation by binding to the promoters of genes involved in various human cancers, thereby regulating the expression of various cancer related genes and influencing the signaling pathways related to tumor development at the genomic level. Therefore, it plays a critical role in cell activities, such as cell growth, proliferation, migration, apoptosis, and metabolism. In addition, DHX33 was found to sense pathogenic double-stranded RNA molecules, and play an important role in innate immunity of host cells. As a very important gene regulating cellular growth, DHX33 was found to be highly expressed in many cancers, including lung cancer, lymphoma, glioblastoma, breast cancer, colorectal cancer, liver cancer, etc. The initiation and progression of various cancers depend on the high expression of DHX33 protein. Genetic knockout of DHX33 can significantly suppress RAS oncogene-driven lung cancer development. Both in vitro and in vivo experiments have demonstrated that inhibition of the DHX33 protein greatly suppresses the initiation and progression of various cancers, including breast cancer, colorectal cancer, glioblastoma, and lymphoma. Since the protein function of DHX33 depends on its helicase activity, and the helicase activity-deficient mutant of DHX33 does not have the function of DHX33 protein, it cannot compensate the defect that was caused by the deletion of wild-type DHX33 protein.

The inventor has identified a variety of compounds capable of inhibiting the RNA helicase activity of DHX33 (such as, 1-(3-cyano-5-methylthiophen-2-yl)-N-(6-methoxy-1H-benzo[d]imidazol-2-yl)-2,5-dimethyl-1H-pyrrole-3-carboxamide), and has verified that these compounds could significantly suppress the growth and proliferation of cancer cells both in vitro and in vivo.

### SUMMARY

An objective of the present invention is to provide different crystal forms of 1-(3-cyano-5-methylthiophen-2-yl)-N-(6-methoxy-1H-benzo[d]imidazol-2-yl)-2,5-dimethyl-1H-pyrrole-3-carboxamide (hereinafter referred to as “compound of Formula (1)”), a pharmaceutical composition including the crystal form and the use.

A structural formula of the compound of Formula (1) is as follows:

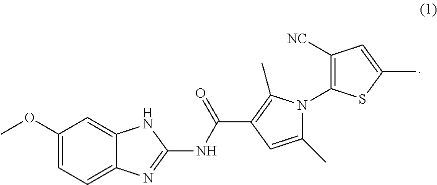

In a first aspect, the present invention provides an anhydrous crystal form A of the compound of Formula (1), where an X-ray powder diffraction (XRPD) pattern of the crystal form A has characteristic diffraction peaks at the following 2θ angles: 5.36±0.2°, 12.17±0.2° and 10.85±0.2°.

In some embodiments, the XRPD pattern of the anhydrous crystal form A of the compound of Formula (1) further has characteristic diffraction peaks at one or more of the following 2θ angles of 14.26±0.2°, 9.12±0.2° and 8.26±0.2°.

In some embodiments, the XRPD pattern of the anhydrous crystal form A of the compound of Formula (1) further has characteristic diffraction peaks at one or more of the following 2θ angles of 19.77±0.2°, 20.03±0.2° and 22.17±0.2°.

The present invention further provides a preparation method for anhydrous crystal form A of the compound of Formula (1), including any one of the following methods:
(1) suspending the compound of Formula (1) in a solvent and stirring at 5-35° C. for 1-3 d to obtain a crystal wet sample, which is a crystal form B; and drying the crystal form B at room temperature to convert it into the anhydrous crystal form A; where the solvent is selected from ethanol or a mixed solvent of ethyl acetate and isopropanol;
(2) suspending the compound of Formula (1) in a solvent and stirring at 4-8° C. for 14-18 d, and isolating a resulting solid to obtain the anhydrous crystal form A, where the solvent is selected from methanol, a mixed solvent of acetone and water, a mixed solvent of ethyl acetate and isopropanol, a mixed solvent of methyl tert-butyl ether and methyl ethyl ketone, a mixed solvent of 2-methyltetrahydrofuran and n-heptane, a mixed solvent of tetrahydrofuran and isopropyl acetate, and a mixed solvent of cyclopentyl methyl ether and tetrahydrofuranyl;
(3) suspending the compound of Formula (1) in a solvent and stirring at room temperature for 14-18 d, and then isolating a resulting solid to obtain the anhydrous crystal form A; where the solvent is selected from ethanol, isopropyl acetate, a mixed solvent of dichloromethane and methanol, a mixed solvent of methyl ethyl ketone and methylcyclohexane, a mixed solvent of 1,4-dioxane and toluene, a mixed solvent of isopropanol and water, a mixed solvent of methyl isobutyl ketone and acetonitrile, and a mixed solvent of acetone and ethyl acetate; and
(4) converting the anhydrous crystal form C into the anhydrous crystal form A by air-drying at room temperature.

In some embodiments, the compound of Formula (1) is suspended in a solvent and stirred at 5-35° C. for about 1-3 d to obtain a crystal wet sample, which is a crystal form B, and the crystal form B is air-dried at room temperature for 1 d and converted into an anhydrous crystal form A. The solvent is selected from a mixed solvent of ethanol, ethyl acetate and isopropanol (with a volume ratio of 1:1).

In some embodiments, the compound of Formula (1) is dissolved in a solvent and filtered to obtain a clear solution. A vial containing the clear solution is sealed with a perforated sealing film and allowed to evaporate slowly at room temperature, and the obtained solid is collected to obtain the anhydrous crystal form A. The solvent is selected from a mixed solvent of acetone and acetonitrile (with a volume ratio of 3:1); a mixed solvent of ethyl acetate, tetrahydrofuran, and water (with a volume ratio of 1:1); a mixed solvent of dichloromethane and cyclopentyl methyl ether (with a volume ratio of 1:1); and a mixed solvent of toluene and tetrahydrofuran (with a volume ratio of 2:1).

In some embodiments, the compound of Formula (1) is suspended in a solvent and stirred at 4-8° C. for about 14-18 d, and a resulting solid is isolated to obtain the anhydrous crystal form A. The solvent is selected from methanol, a mixed solvent of acetone and water (with a volume ratio of 1:1), a mixed solvent of ethyl acetate and isopropanol (with a volume ratio of 1:2), a mixed solvent of methyl tert-butyl ether and methyl ethyl ketone (with a volume ratio of 1:1), a mixed solvent of 2-methyltetrahydrofuran and n-heptane (with a volume ratio of 1:1), a mixed solvent of tetrahydrofuran and isopropyl acetate (with a volume ratio of 1:4), and a mixed solvent of cyclopentyl methyl ether and tetrahydrofuranyl (with a volume ratio of 4:1).

In some embodiments, the compound of Formula (1) is suspended in a solvent and stirred at room temperature for about 14-18 d, and a resulting solid is isolated to obtain the anhydrous crystal form A. The solvent is selected from ethanol, isopropyl acetate, and a mixed solvent of dichloromethane and methanol (with a volume ratio of 1:4), a mixed solvent of methyl ethyl ketone and methylcyclohexane (with a volume ratio of 1:2), a mixed solvent of 1,4-dioxane and toluene (with a volume ratio of 1:3), a mixed solvent of isopropanol and water (with a volume ratio of 1:1), a mixed solvent of methyl isobutyl ketone and acetonitrile (with a volume ratio of 1:1), and a mixed solvent of acetone and ethyl acetate (with a volume ratio of 1:5).

In some embodiments, the compound of Formula (1) is suspended in a solvent and stirred at 50° C. for about 7 d, and then stirred at 5° C. for about 11 d, and a resulting solid is isolated to obtain the anhydrous crystal form A. The solvent is selected from a mixed solvent of cyclopentyl methyl ether and ethanol (with a volume ratio of 1:1), a mixed solvent of tetrahydrofuran and water (with a volume ratio of 1:6) and a mixed solvent of 1,4-dioxane and n-hexane (with a volume ratio of 1:3).

In some embodiments, the compound of Formula (1) is suspended in a solvent, heated to 50° C. and equilibrated for about 2 h, and then filtered to obtain a filtrate, the filtrate is cooled slowly to room temperature in a water bath, and a resulting solid is isolated to obtain the anhydrous crystal form A. The solvent is selected from methanol.

In some embodiments, the compound of Formula (1) is dissolved in a good solvent and filtered to obtain a clear solution. a vial containing the clear solution is then placed into another vial containing a volatile anti-solvent, and the vial is sealed with a cap and maintained at room temperature for about 1-3 d, and a resulting solid is isolated to obtain the anhydrous crystal form A. The good solvent is selected from a mixed solvent of 1,4-dioxane and toluene (with a volume ratio of 4:1), a mixed solvent of methyl ethyl ketone and acetonitrile (with a volume ratio of 4:1), and a mixed solvent of chloroform and methyl isobutyl ketone (with a volume ratio of 2:1). The anti-solvent is selected from n-pentane and n-heptane.

In some embodiments, the compound of Formula (1) is suspended in a solvent and stirred under temperature cycling conditions, and a resulting solid is isolated to obtain the anhydrous crystal form A. The solvent is selected from a mixed solvent of isopropyl acetate and cyclopentyl methyl ether (with a volume ratio of 1:1), a mixed solvent of methanol and methyl ethyl ketone (with a volume ratio of 4:1), a mixed solvent of ethyl acetate and chloroform (with a volume ratio of 5:1), a mixed solvent of tetrahydrofuran and n-heptane (with a volume ratio of 1:5), a mixed solvent of 1,4-dioxane and methyl tert-butyl ether (with a volume ratio of 1:3), and a mixed solvent of methyl isobutyl ketone and methylcyclohexane (with a volume ratio of 1:1). The temperature cycling condition involves equilibrating at 50° C. for about 120 min, then cooling to 5° C. at a rate of 0.1° C./min, equilibrating at 5° C. for about 120 min, and repeating the cycle twice.

In a second aspect, the present invention provides an anhydrous crystal form C of the compound of Formula (1), where an X-ray powder diffraction (XRPD) pattern of the crystal form C has characteristic diffraction peaks at the following 2θ angles: 5.73±0.2° 8.16±0.2° and 12.94±0.2°.

In some embodiments, the XRPD pattern of the anhydrous crystal form C of the compound of Formula (1) further has characteristic diffraction peaks at one or more of the following 2θ angles of 11.55±0.2°, 15.10±0.2° and 18.37±0.2°.

In some embodiments, the XRPD pattern of the anhydrous crystal form C of the compound of Formula (1) further has characteristic diffraction peaks at one or more of the following 2θ angles of 13.29±0.2°, 8.73±0.2° and 23.61±0.2°.

The present invention further provides a preparation method for anhydrous crystal form C of the compound of Formula (1), including any one of the following methods:
(1) suspending the compound of Formula (1) in a solvent and stirring at 5-35° C. for 1-3 d to obtain a crystal wet sample, which is a crystal form B; and converting the crystal form B into the anhydrous crystal form C by drying at 50-80° C.; where the solvent is selected from ethanol or a mixed solvent of ethyl acetate and isopropanol.
(2) suspending the compound of Formula (1) in a solvent and stirring at 4-8° C. for 3 d, and isolating a resulting solid to obtain the anhydrous crystal form C; where the solvent is selected from a mixed solvent of ethyl acetate and isopropanol;
(3) suspending the compound of Formula (1) in a solvent and stirring at room temperature for 14-18 d, and isolating a resulting solid to obtain the anhydrous crystal form C; where the solvent is selected from a mixed solvent of acetonitrile and water.

In some embodiments, the compound of Formula (1) is suspended in a solvent and stirred at 5-35° C. for about 1-3 d to obtain a crystal wet sample, which is a crystal form B, The crystal form B is dried at 50-80° C. for 2-12 h and converted into an anhydrous crystal form C. The solvent is selected from a mixed solvent of ethanol, ethyl acetate and isopropanol (with a volume ratio of 1:1).

In some embodiments, the compound of Formula (1) is suspended in a solvent and stirred at 4-8° C. for about 3 d, and a resulting solid is isolated to obtain an anhydrous crystal form C. The solvent is selected from a mixed solvent of ethyl acetate and isopropanol (with a volume ratio of 1:1).

In some embodiments, the compound of Formula (1) is suspended in a solvent and stirred at 4-8° C. for about 18 d, and a resulting solid is isolated to obtain an anhydrous crystal form C. The solvent is selected from a mixed solvent of dichloromethane and acetonitrile (with a volume ratio of 1:3).

In some embodiments, the compound of Formula (1) is suspended in a solvent, heated to 50° C. and equilibrated for about 2 h, and then filtered to obtain a filtrate, the filtrate is cooled slowly to room temperature in a water bath, and a resulting solid is isolated to obtain an anhydrous crystal form C. The solvent is selected from a mixed solvent of acetonitrile and water (with a volume ratio of 3:1).

In some embodiments, the compound of Formula (1) is suspended in a solvent and stirred under temperature cycling conditions, and a resulting solid is isolated to obtain an anhydrous crystal form C. The solvent is selected from acetonitrile. The temperature cycling condition involves equilibrating at 50° C. for about 120 min, then cooling to 5° C. at a rate of 0.1° C./min, equilibrating at 5° C. for about 120 min, and repeating the cycle twice.

In some embodiments, the compound of Formula (1) is suspended in a solvent and stirred at room temperature for about 14-18 d, and a resulting solid is isolated to obtain an anhydrous crystal form C. The solvent is selected from a mixed solvent of acetonitrile and water (with a volume ratio of 1:1).

In some embodiments, the compound of Formula (1) is suspended in a solvent and stirred at 4-8° C. for about 18 d, and a resulting solid is isolated to obtain a crystal form D, and the crystal form D is air-dried at room temperature for 1 d and converted into an anhydrous crystal form C. The solvent is selected from a mixed solvent of toluene and ethanol (with a volume ratio of 1:1).

In a third aspect, the present invention provides a pharmaceutical composition, including at least one of the above crystal forms, and one or more pharmaceutically acceptable carriers.

In a fourth aspect, the present invention provides use of the above crystal forms or the pharmaceutical composition in the preparation of a drug for preventing and/or treating a disease or condition mediated by DHX33.

In a fifth aspect, the present invention provides a method for preventing and/or treating a disease or condition mediated by DHX33, including the following steps: administering a preventive and/or a therapeutically effective amount of the above crystal form or the pharmaceutical composition to an individual in need thereof; and preferably, the disease is selected from cancer, viral infection and inflammation mediated by DHX33.

The present invention is not limited to the specific embodiments described herein; it should also be understood that the terms used herein are for description purposes only and are not intended to limit the specific embodiments.

### Terms and Definitions

Unless otherwise defined, all technical and scientific terms used in the present invention have the same meanings as commonly understood by those ordinarily skilled in the art to which the present invention belongs. In the event of any conflict, the definitions provided in the present invention shall prevail. When a quantity, a concentration, or other value or parameter is expressed in the form of a range, a preferred range, or a preferred upper numerical limit and a preferred lower numerical limit, it should be understood that it specifically discloses any range formed by combining an upper limit or preferred numerical limit with a lower limit or preferred numerical limit of any pair of range, regardless of whether the range is specifically stated. Unless otherwise stated, the numerical ranges listed in the present invention are intended to include the endpoints of the range, as well as all integers and fractional (decimal) values within the ranges.

The term “about”, when being used in conjunction with a numerical variable, generally refers to the situation that a value of the variable and all values of the variable fall within an experimental error (for example, within a 95% confidence interval for the mean) or within ±10% of a specified value, or within a wider range.

The phrase “comprising” or synonymous similar expressions such as “including,” “containing,” and “having” are open-ended, and do not exclude additional, unrecited elements, steps, or ingredients. The phrase “consisting of” excludes any elements, steps, or ingredients not explicitly specified. The phrase “consisting essentially of” limits the scope to the specified elements, steps, or ingredients, along with optional elements, steps, or ingredients that do not materially affect the basic and novel characteristics of the claimed subject matter. It should be understood that the phrase “comprising” encompasses the phrases of both “consisting essentially of” and “consisting of.”

The term “X-ray powder diffraction pattern (XRPD pattern)” refers to an experimentally observed diffraction pattern or the parameters, data, or values derived therefrom. XRPD patterns are typically characterized by peak positions (x-axis) and/or peak intensities (y-axis).

The term “diffraction angle” or “20” refers to the peak position measured in degrees (°) based on the setting in the X-ray diffraction experiments, and is usually represented as the x-axis unit in diffraction patterns. The reflection is diffracted when the incident beam forms an angle θ with a certain crystal lattice plane, and the experiment is configured to record the reflected beam at an angle of 2θ. It should be understood that the specific 2θ value of a specific crystal form mentioned in the present invention is intended to represent the 2θ values (expressed in degrees) measured using the X-ray diffraction experimental conditions described herein.

It should be noted that in the powder X-ray diffraction spectrum, peak positions or relative peak intensities may vary due to factors such as measuring instruments, measuring methods/conditions. For any specific crystal form, there may be an error in peak positions, and the measurement error of the 2θ value may be ±0.2°. Therefore, when identifying each crystal form, the error should be taken into account, and values within the error should fall within the scope of the present invention.

For the same crystal form, a position of endothermic peak in differential scanning calorimetry (DSC) may vary due to factors such as measuring instruments, measuring methods/conditions. For any specific crystal form, there may be an error in the position of endothermic peak, and the error may be ±5° C. or ±3° C. Therefore, when identifying each crystal form, the error should be taken into account, and values within the error should fall within the scope of the present invention.

For the same crystal form, an onset position of weight loss temperature in thermogravimetric analysis (TGA) may vary due to factors such as measuring instruments, measuring methods/conditions. For any specific crystal form, there may be an error in the position of weight loss temperature, and the error may be ±5° C. or ±3° C. Therefore, when identifying each crystal form, the error should be taken into account, and values within the error should fall within the scope of the present invention.

It should be understood that different types of equipment or different test conditions may produce slightly different XRPD patterns and characteristic peaks or different DSC spectra and characteristic peaks. The specific values provided should not be considered as absolute values.

The term “room temperature” refers to 20° C. 5° C.

The term “prevention” refers to prophylactic administration to reduce the likelihood of or delay the onset of a disease or symptom.

The term “treatment” is intended to alleviate or eliminate the disease state or condition that is treated. It should also be understood that the treatment of the disease state or condition includes not only complete eradication, but also incomplete treatment but the achievement of some biologically or medically relevant outcomes.

The term “pharmaceutically acceptable” refers to the compounds, materials, compositions, and/or crystal forms that, within the scope of sound medical judgment, are suitable for contact with human or animal tissues without excessive toxicity, irritation, allergic reaction, or other problems or complications, and that have an appropriate benefit-to-risk ratio. The term “pharmaceutically acceptable carrier” refers to an inert substance that is administered together with the active ingredient and facilitates the administration of the active ingredient, including but not limited to any glidant, sweetener, diluent, preservative, dye/colorant, flavor enhancer, surfactant, wetting agent, dispersing agent, disintegrant, suspending agent, stabilizer, isotonic agent, solvent, or emulsifier that have been approved by the National Medical Products Administration (NMPA) for human or animal (such as livestock) use.

The above pharmaceutical compositions may exert systemic and/or local efficacy. For this purpose, they may be administered through appropriate routes, such as parenteral, topical, intravenous, oral, subcutaneous, intra-arterial, intradermal, transdermal, rectal, intracranial, intraperitoneal, intranasal, intramuscular routes, or as inhalation formulations.

The above administration routes may be realized through suitable dosage forms. The dosage forms available for use in the present invention include but are not limited to: tablets, capsules, lozenges, pastilles, powders, sprays, creams, ointments, suppositories, gels, pastes, lotions, aqueous suspensions, injectable solutions, elixirs, syrups, and the like.

When being administered orally, the pharmaceutical compositions may be prepared into any orally acceptable dosage forms, including but not limited to tablets, capsules, aqueous solutions, aqueous suspensions, and the like.

The term “disease or condition mediated by DHX33” refers to a disease pathogenesis of which includes at least a part of factors related to DHX33, such as cancers, viral infections, and inflammatory conditions.

The term “effective amount” refers to a dosage sufficient to elicit a desired biological or medical response in a cell, tissue, organ, or organism (such as an individual) and sufficient to achieve the desired preventive and/or therapeutic effect.

The therapeutically effective amount of the crystal form described in the present invention ranges from approximately 0.0001 to 20 mg/Kg body weight per day, for example, from 0.001 to 10 mg/Kg body weight per day.

The dosage frequency of the crystal form described in the present invention is determined by the needs of an individual patient, for example, once or twice daily, or more times a day. Administration may be intermittent, for example, during a period of several days, the patient receives a daily dose of the crystal form, followed by a period of several days or more days during which the patient does not receive a daily dosage of the crystal form.

### BRIEF DESCRIPTION OF THE DRAWINGS

FIG. 1 is an X-ray powder diffraction (XRPD) pattern of a crystal form E of a compound of Formula (1).

FIG. 2 is a thermogravimetric analysis (TGA) diagram of a crystal form E.

FIG. 3 is a differential scanning calorimetry (DSC) diagram of a crystal form E.

FIG. 4 is a ¹H NMR spectrum of a crystal form E.

FIG. 5 is an XRPD comparison diagram of an anhydrous crystal form A of Compound (1), a crystal form B, an anhydrous crystal form C, and a crystal form D.

FIG. 6 is an XRPD comparison diagram of a wet sample and a dried sample of a preparation method of an anhydrous crystal form A.

FIG. 7 is an XRPD diagram of an anhydrous crystal form A.

FIG. 8 is a TGA diagram of an anhydrous crystal form A.

FIG. 9 is a DSC diagram of an anhydrous crystal form A.

FIG. 10 is a ¹H NMR spectrum of an anhydrous crystal form A.

FIG. 11 is an XRPD comparison diagram of an anhydrous crystal form A before and after heating.

FIG. 12 is a TGA diagram of an anhydrous crystal form A before and after heating.

FIG. 13 is a VT-XRPD pattern of an anhydrous crystal form A.

FIG. 14 is an XRPD diagram of an anhydrous crystal form C.

FIG. 15 is an XRPD comparison diagram of a wet sample and a dried sample in one preparation method of an anhydrous crystal form C.

FIG. 16 is a TGA diagram of an anhydrous crystal form C.

FIG. 17 is a DSC diagram of an anhydrous crystal form C.

FIG. 18 is a ¹H NMR spectrum of an anhydrous crystal form C.

FIG. 19 is an XRPD comparison diagram of a wet sample and a dried sample in another preparation method of an anhydrous crystal form C.

FIG. 20 is an XRPD diagram of a crystal form B.

FIG. 21 is an XRPD comparison diagram of a crystal form B before and after storage at room temperature.

FIG. 22 is an XRPD diagram of a crystal form D.

FIG. 23 is an XRPD comparison diagram of a crystal form D before and after storage at room temperature.

FIG. 24 is an XRPD comparison diagram of suspension competition between an anhydrous crystal form A and an anhydrous crystal form C.

FIG. 25 is an XRPD comparison diagram of an anhydrous crystal form A before and after solid-state stability test.

FIG. 26 is an XRPD comparison diagram of an anhydrous crystal form C before and after solid-state stability test.

FIG. 27 is an XRPD comparison diagram of an anhydrous crystal form A before and after high-temperature stability test.

FIG. 28 is an XRPD comparison diagram of an anhydrous crystal form C before and after high-temperature stability test.

FIG. 29 is DVS test diagram of an anhydrous crystal form A.

FIG. 30 is an XRPD comparison diagram of an anhydrous crystal form A before and after DVS test.

FIG. 31 is DVS test diagram of an anhydrous crystal form C.

FIG. 32 is an XRPD comparison diagram of an anhydrous crystal form C before and after DVS test.

### DETAILED DESCRIPTION OF THE EMBODIMENTS

The intermediate compounds of the present invention can be prepared using various synthetic methods known to those skilled in the art, including the specific embodiments stated below, the embodiments formed by combining them with other chemical synthesis methods, and equivalent substitution methods recognized by those skilled in the art, and preferred embodiments include but are not limited to those described in the examples of the present invention.

The chemical reactions of the specific embodiments of the present invention are carried out in suitable solvents, which must be appropriate for the chemical changes, and the required reagents and materials in the present invention. In order to obtain the compounds of the present invention, it is sometimes necessary for those skilled in the art to modify or select synthesis steps or reaction processes based on the existing embodiments.

The present invention will be described in detail below through the embodiments, which are not intended to limit the scope of the present invention.

### X-Ray Powder Diffraction (XRPD)

### XRPD Test Parameters

X-ray powder diffraction (XRPD)

| | |
|---|---|
| Model | Bruker D8 Advance |
| No. | CA312 |
| Technical indicators | Kα radiation (40 kV, 40 mA) with a |
| | wavelength of 1.54 Å on a copper |
| | target, θ-2θ goniometer, nickel |
| | filter, SSD160-2 detector |
| Acquisition software | DIFFRAC.MEA.CENTER |
| Calibration material | Corundum (Al₂O₃) |
| Analysis software | MDI Jade 6 |
| Testing angle | 3-40°2θ |
| Step size | 0.02°2θ |
| Speed | 0.1 s/step |
| Sample amount under | >1 mg |
| testing | |
| Remark: | Remarks: Unless otherwise specified, |
| | samples are not ground before testing |

### Thermogravimetric Analysis (TGA) and Differential Scanning Calorimetry (DSC)

### TGA and DSC Testing Parameters

| | | | |
|---|---|---|---|
| Instruments | Model | TGA 2 | DSC 3 |
| | No. | CA201 | CA200 |
| | Control software | STARe software | STARe software |
| | Analysis | STARe software | STARe software |
| | software | | |
| | Sample tray | Alumina crucible | Aluminum crucible |
| | | | (with perforated lid) |
| Parameters | Sample amount | 1 mg-10 mg | 0.5 mg-5 mg |
| | under testing | | |
| | Protective gas | Nitrogen | Nitrogen |
| | Gas flow rate | 50 mL/min | 50 mL/min |
| | Common testing | Segment 1 | Segment 1 |
| | method | Starting temperature 30.0° C. | Starting temperature 30° C. |
| | | Ending temperature 350.0° C. | Ending temperature 300° C. |
| | | Heating rate 10.0 k/min | Heating rate 10.0 k/min |

### Nuclear Magnetic Resonance Spectroscopy (¹H NMR)

### ¹H NMR Testing Parameters

Nuclear magnetic resonance spectrometer (NMR)

| | | | | |
|---|---|---|---|---|
| Instruments | Model | Bruker AVANCE | Bruker AVANCE | Bruker AVANCE |
| | | III 400 MHZ | NEO 400 MHZ | NEO 400 MHZ |
| | No. | NMR-002 | NMR-003 | NMR-004 |
| | Testing type | Nuclear magnetic | Nuclear magnetic | Nuclear magnetic |
| | | resonance | resonance | resonance |
| | | spectroscopy | spectroscopy | spectroscopy |

| | |
|---|---|
| Parameters | full-spectrum excitation, a spectral width of 20 ppm using a single pulse, and a |
| | 30° pulse angle with 8 scans, digital quadrature detection, a temperature controlled |
| | at 298 K. Unless otherwise specified, DMSO-d6 was used as a solvent. |

### High-Performance Liquid Chromatography (HPLC)

### HPLC Testing Parameters

| Instruments | High-performance liquid chromatography | | |
|---|---|---|---|
| Chromatographic column | Phenomenex Gemini, | | |
| | 4.6*150 mm*5 μm | | |
| Mobile phase | Mobile phase A: 0.1% formic acid | | |
| | aqueous solution | | |
| | Mobile phase B: MeOH | | |
| Flow rate | 1.0 mL/min | | |
| Acquisition time | 37 min | | |
| Measured wavelength | 246 nm | | |
| Instruments | High-performance liquid chromatography | | |
| Column temperature | 30° C. | | |
| Sample injector temperature | Room temperature | | |
| Sample injection volume | 20.0 or 2.0 μL | | |
| Thinner | ACN | | |
| | Time (min) | A (%) | B (%) |
| Gradient elution table | 0.00 | 65 | 35 |
| | 3.00 | 65 | 35 |
| | 10.00 | 48 | 52 |
| | 25.00 | 20 | 80 |
| | 30.00 | 20 | 80 |
| | 30.10 | 65 | 35 |
| | 37.00 | 65 | 35 |

### Dynamic Vapor Sorption (DVS)

### DVS Testing Parameters

DVS analyzer from UK SMS

| | |
|---|---|
| Model | DVS Intrinsic |
| No. | DVS-001 (CA357) |
| Method | 8. Equilibrate at 25° C. |
| parameters | 9. Humidity 0% |
| | 10. Isothermal period: 360 min |
| | 11. Experiment stops when a weight change exceeds |
| | the limit: (%/min) < 0.00200 |
| | 12. Stepwise humidity control 0%-90% |
| | 13. Experiment stops when a weight change exceeds |
| | the limit: (%/min) <0.00200 |
| | 14. Stepwise humidity control 90%-0% |

### LIST OF SOLVENT ABBREVIATIONS

| Abbreviation | Full name |
|---|---|
| H₂O | Water |
| MeOH | Methanol |
| EtOH | Ethanol |
| IPA | Isopropanol |
| TFE | Trifluoroethanol |
| Acetone | Acetone |
| MIBK | Methyl isobutyl ketone |
| EtOAc | Ethyl acetate |
| IPAc | Isopropyl acetate |
| MTBE | Methyl tert-butyl ether |
| MEK | Methyl Ethyl Ketone |
| Anisole | Methyl phenyl ether |
| n-Hexane | n-Hexane |
| n-Pentane | n-Pentane |
| ACN | Acetonitrile |
| CH₂Cl₂ | Dichloromethane |
| THF | Tetrahydrofuran |
| 2-MeTHF | 2-Methyltetrahydrofuran |
| 1,4-Dioxane | 1,4-Dioxane |
| Toluene | Toluene |
| HEP | Heptane |
| DMSO | Dimethyl sulfoxide |
| DMF | N,N-Dimethylformamide |
| NMP | N-methylpyrrolidone |
| CPME | Cyclopentyl methyl ether |
| CHCl₃ | Chloroform |
| MCH | Methylcyclohexane |
| Xylene | Xylene |

### Example 1: Preparation Method for Compound of Formula (1): 1-(3-cyano-5-methylthiophen-2-yl)-N-(6-methoxy-1H-benzo[d]imidazol-2-yl)-2,5-dimethyl-1H-pyrrole-3-carboxamide

### (1) Preparation method for Compound 3 (ethyl 2-acetyl-4-oxopentanoate)

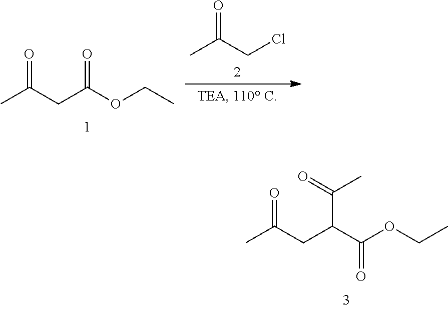

Compound 1 (ethyl acetoacetate) (5 g, 38.42 mmol, 1.0 eq) was dissolved in triethylamine (75 mL), and Compound 2 (chloroacetone) (3.5 g, 38.42 mmol, 1.0 eq) was added to obtain a reactant; the reactant was reacted at 110° C. for 2 h under the conditions of nitrogen protection; the reactant was concentrated to obtain a residue, the residue was dissolved in water (100 mL), and extracted twice with dichloromethane (50 mL each time); an organic layer was washed with brine, dried with anhydrous sodium sulfate, filtered, and concentrated, and the residue was purified by flash column chromatography (petroleum ether/ethyl acetate=100/1 to 50/1 to 20/1) to obtain colorless oily Compound 3 (ethyl 2-acetyl-4-oxopentanoate) (1.3 g, yield: 18.3%). MS (ESI) m/z: 187 [M+H⁺]. TLC PE/EA(2/1); R_{f} (Compound 1)=0.6; R_{f} (Compound 3)=0.4.

### (2) Preparation Method for Compound 5 (1-(3-cyano-5-methylthiophen-2-yl)-2,5-dimethyl-1H-pyrrole-3-carboxamide)

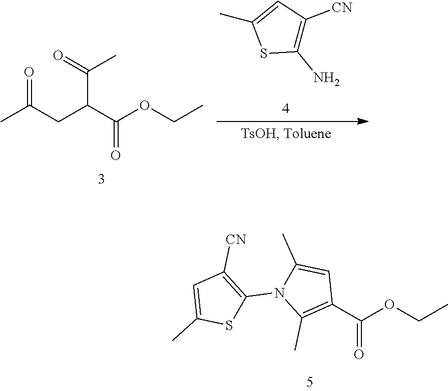

Compound 3 (ethyl 2-acetyl-4-oxopentanoate) (1 g, 7.23 mmol, 1.0 eq) was dissolved in toluene (20 mL), and Compound 4 (2-amino-3-cyano-5-methylthiophene) (1.6 g, 8.68 mmol, 1.2 eq) and p-toluenesulfonic acid (249 mg, 1.45 mmol, 0.2 eq) were added to obtain a reactant; the reactant was stirred at 110° C. for 16 h to obtain a solid; the solid was filtered and concentrated, and a resulting residue was purified by flash column chromatography (petroleum ether/ethyl acetate=50/1 to 30/1) to obtain yellow oily Compound 5 (1-(3-cyano-5-methylthiophen-2-yl)-2,5-dimethyl-1H-pyrrole-3-carboxamide (860 mg, yield: 41%). MS (ESI) m/z: 289 [M+H⁺]. TLC:Petroleum ether/ethyl acetate (10/1); R_{f} (Compound 3)=0.2; R_{f} (Compound 5)=0.4.

### (3) Preparation Method for Compound of Formula (1)

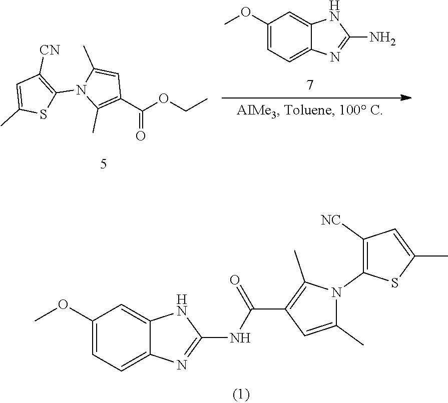

Trimethylaluminum (0.15 mL, 0.306 mmol, 1.0 eq, 2 M in toluene) was added to Compound 5 (1-(3-cyano-5-methylthiophen-2-yl)-2,5-dimethyl-1H-pyrrole-3-carboxamide) (50 mg, 0.306 mmol, 1.0 eq) and Compound 7 (5-methoxy-1H-benzimidazol-2-amine) (88 mg, 0.306 mmol, 1.0 eq) dissolved in 1 mL of toluene to obtain a reactant; the reactant was stirred at 100° C. for 16 h to obtain a mixture; the mixture was cooled to room temperature, and quenched with methanol (10 mL), and a pH value of the mixture was adjusted to 3 with 3 M hydrochloric acid; the mixture was diluted with water (30 mL), and extracted with ethyl acetate three times, 20 mL each time; an organic layer was washed with brine, dried with anhydrous sodium sulfate, filtered, and concentrated, and the resulting residue was purified by preparative high-pressure liquid chromatography (Prep-HPLC, acetonitrile/water, containing 0.1% formic acid) to obtain the compound of Formula (1) (5 mg, yield: 4%), which existed in the form of a weakly crystalline solid in crystal form E. MS (ESI) m/z: 406 [M+H⁺]. ¹H NMR (400 MHz, DMSO-d₆): δ 12.04 (s, 1H), 11.20 (s, 1H), 7.32 (s, 1H), 7.29 (s, 1H), 6.98 (s, 1H), 6.89 (s, 1H), 6.69 (d, J=7.2 Hz, 1H), 3.73 (s, 3H), 2.47 (s, 3H), 2.38 (s, 3H), 2.04 (s, 3H). ¹H NMR details were shown in FIG. 4.

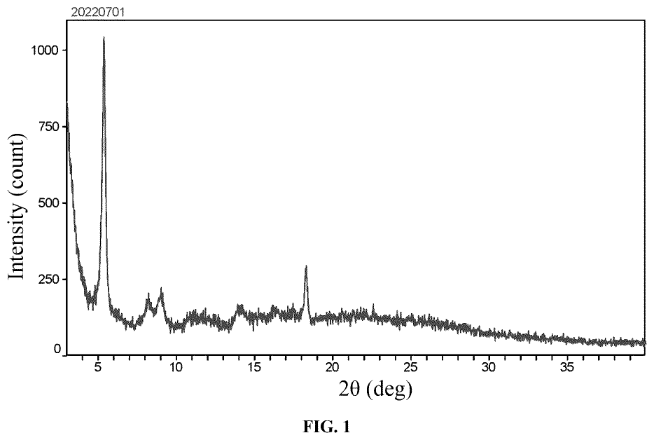

crystal form E has an XRPD pattern substantially as shown in FIG. 1.

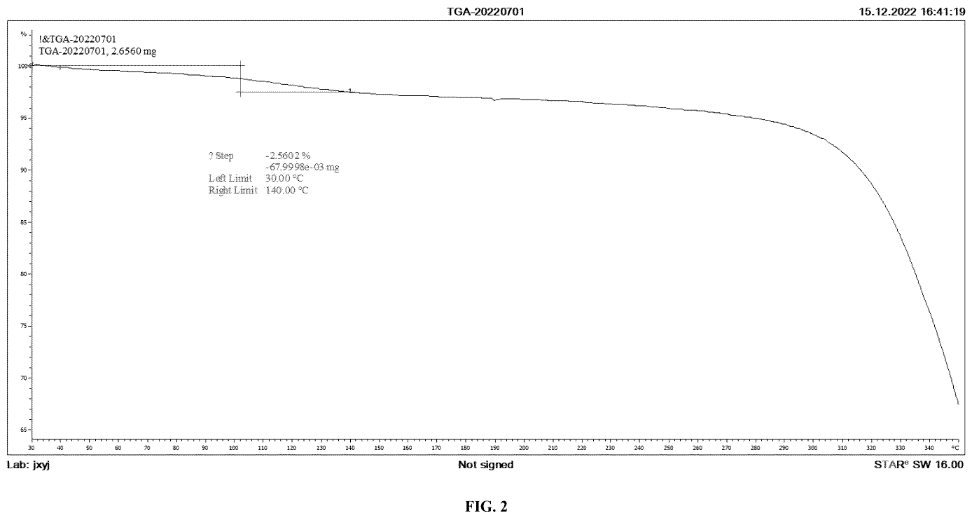

As shown in FIG. 2, a thermogravimetric analysis (TGA) curve of crystal form E indicates a weight loss of about 2.56% when a temperature is raised to about 140° C.

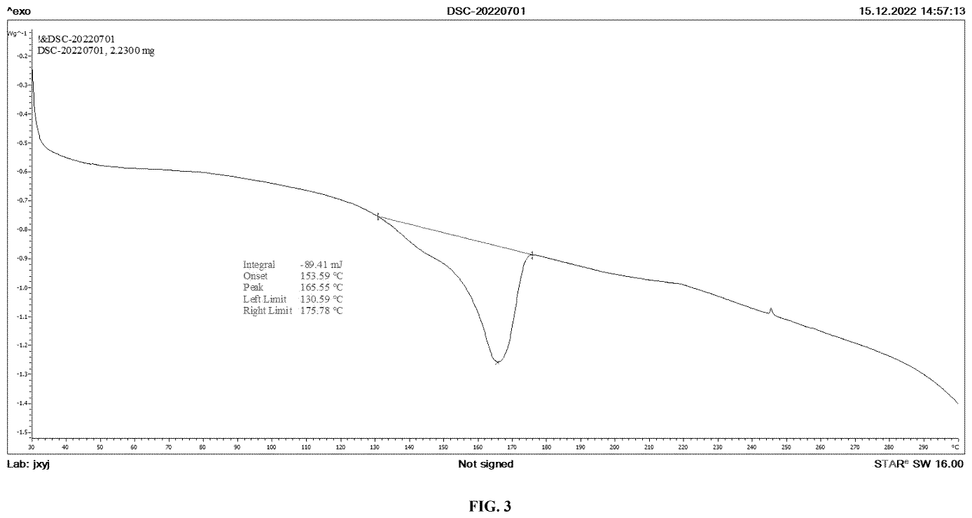

As shown in FIG. 3, a differential scanning calorimetry (DSC) curve of crystal form E exhibits an endothermic peak at about 165.55° C.

### Example 2: Crystal Form of Compound of Formula (1) and Preparation Method Therefor

### Preparation Method for Anhydrous Crystal Form a of Compound of Formula (1)

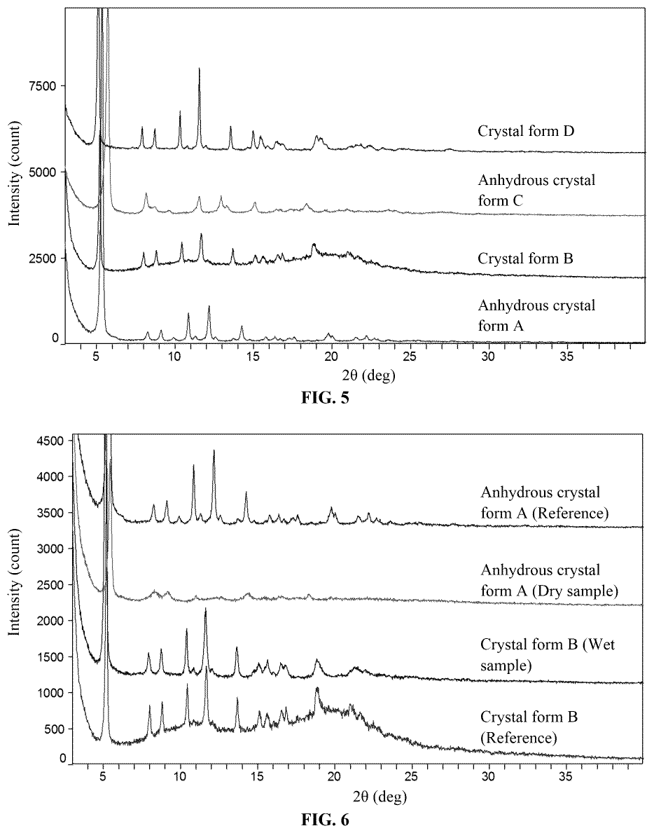

About 199.79 mg of the compound of Formula (1) was weighed and suspended in 5.0 mL of EtOH to obtain a suspension, and the suspension was magnetically stirred at room temperature for 1 d to obtain a crystalline wet solid, which was crystal form B; and the crystal form B was air-dried at room temperature for 1 d and converted into the anhydrous crystal form A. The XRPD comparison of the wet sample and dried sample of the preparation method is shown in FIG. 6.

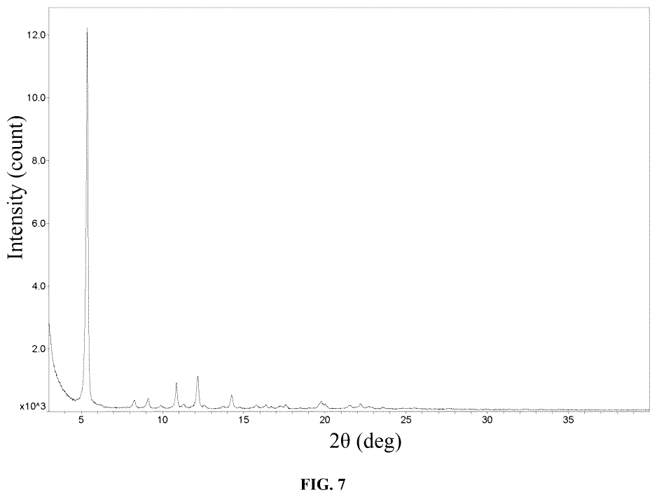

The anhydrous crystal form A has an XRPD pattern substantially as shown in FIG. 7.

The XRPD pattern of the anhydrous crystal form A has characteristic diffraction peaks at 2θ angles listed in Table 1.

TABLE 1

| 2θ | d-spacing | Relative intensity |
|---|---|---|
| (±0.2°) | (Å) | (%) |
| 5.36 | 16.48 | 100.00 |
| 8.26 | 10.69 | 2.00 |
| 9.12 | 9.69 | 2.50 |
| 9.92 | 8.91 | 0.80 |
| 10.85 | 8.15 | 6.60 |
| 11.31 | 7.81 | 1.00 |
| 12.17 | 7.26 | 8.30 |
| 12.58 | 7.03 | 0.90 |
| 13.72 | 6.45 | 0.60 |
| 14.26 | 6.21 | 3.70 |
| 15.76 | 5.62 | 0.90 |
| 16.37 | 5.41 | 1.00 |
| 16.71 | 5.30 | 0.40 |
| 17.28 | 5.13 | 0.60 |
| 17.57 | 5.04 | 1.00 |
| 18.51 | 4.79 | 0.30 |
| 19.77 | 4.49 | 2.00 |
| 20.03 | 4.43 | 1.20 |
| 21.51 | 4.13 | 0.80 |
| 22.17 | 4.01 | 1.20 |
| 22.71 | 3.91 | 0.60 |
| 23.59 | 3.77 | 0.40 |

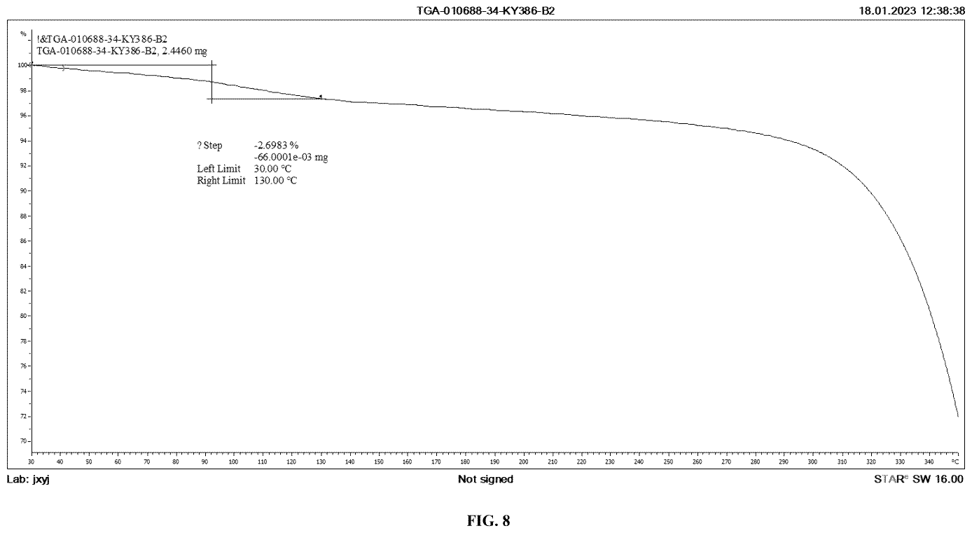

As shown in FIG. 8, a thermogravimetric analysis (TGA) curve of the anhydrous crystal form A indicates a weight loss of about 2.70% when a temperature is raised to about 130° C.

As shown in FIG. 9, a differential scanning calorimetry (DSC) curve of the anhydrous crystal form A exhibits an endothermic peak at about 167.75° C.

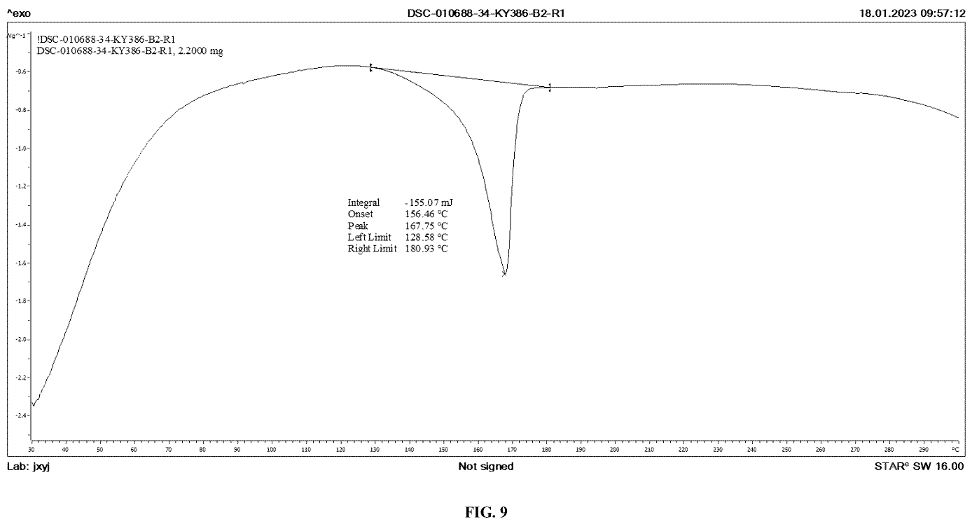

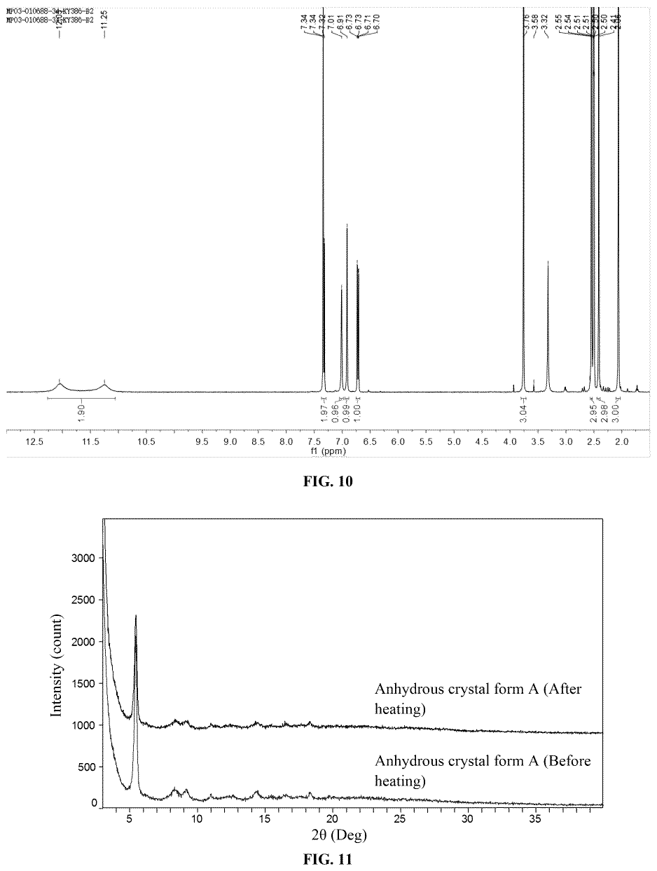

As shown in FIG. 10, the ¹H NMR spectrum of the anhydrous crystal form A indicates no significant solvent residue.

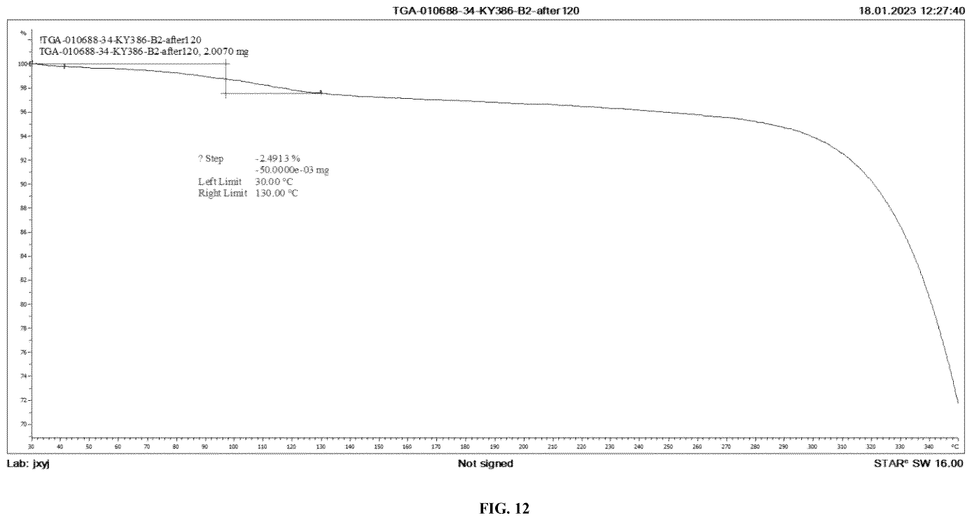

As shown in FIG. 11, in a heating experiment, the anhydrous crystal form A was heated to 120° C. and maintained for 5 min, then cooled to room temperature, and the crystal form remained unchanged during the period. As shown in FIG. 12, a TGA of the heated sample showed a weight loss of 2.49%.

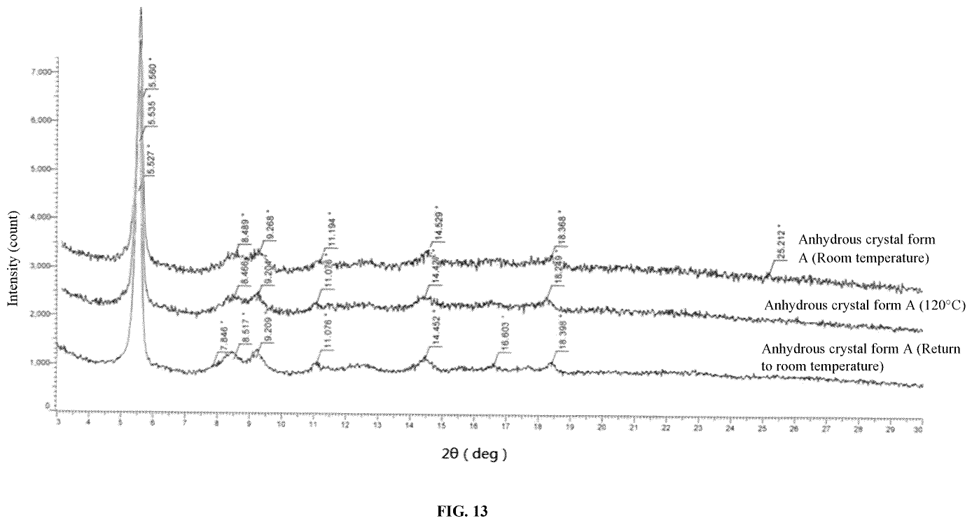

As shown in FIG. 13, in a variable temperature XRPD (VT-XRPD) analysis, the anhydrous crystal form A was heated to 120° C., and an XRPD pattern was detected in situ, showing that the crystal form remained unchanged; and the crystal form was cooled to room temperature, the crystal form remained unchanged.

### Preparation Method for Crystal Form B of Compound of Formula (1)

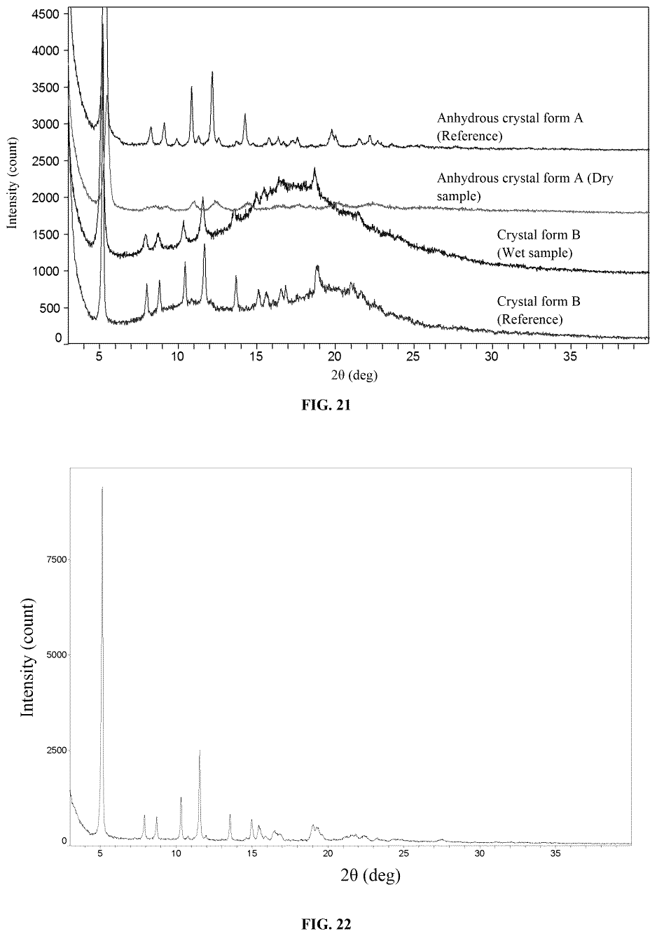

About 199.81 mg of the compound of Formula (1) was weighed and suspended in 5.0 mL of a mixed solvent of methyl ethyl ketone and toluene (with a volume ratio of 1:2) to obtain a suspension, and the suspension was magnetically stirred at 50° C. for 3 d to obtain a crystalline wet solid, which was the crystal form B. crystal form B was a metastable form. crystal form B was air-dried at room temperature for 1 d and converted into the anhydrous crystal form A. The XRPD comparison of the wet sample and dried sample of the preparation method is shown in FIG. 21.

crystal form B has an XRPD pattern substantially as shown in FIG. 20.

The XRPD pattern of crystal form B has characteristic diffraction peaks at 2θ angles listed in Table 2.

TABLE 2

| 2θ | d-spacing | Relative intensity |
|---|---|---|
| (±0.2°) | (Å) | (%) |
| 5.22 | 16.92 | 100.00 |
| 8.01 | 11.04 | 11.00 |
| 8.81 | 10.03 | 10.40 |
| 10.44 | 8.47 | 13.80 |
| 11.67 | 7.58 | 20.80 |
| 13.68 | 6.47 | 11.10 |
| 15.10 | 5.86 | 6.10 |
| 15.62 | 5.67 | 4.70 |
| 16.56 | 5.35 | 5.00 |
| 16.83 | 5.26 | 6.00 |
| 18.88 | 4.70 | 9.30 |
| 21.00 | 4.23 | 4.30 |
| 21.63 | 4.11 | 2.80 |

### Preparation Method for Anhydrous Crystal Form C of Compound of Formula (1)

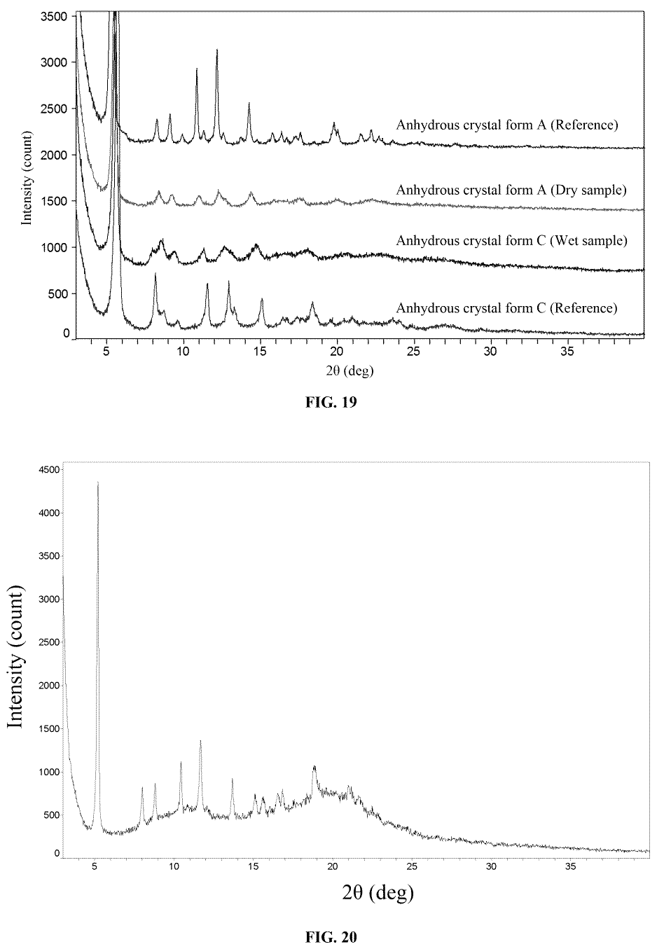

Method 1: about 200.07 mg of the compound of Formula (1) was weighed and suspended in 5.0 mL of a mixed solvent of ethyl acetate and isopropanol (with a volume ratio of 1:1) to obtain a suspension, and the suspension was magnetically stirred at room temperature for 1 d to obtain a crystalline wet solid, which was a crystal form B; and the crystal form B was dried at 50° C. for 4 h and converted into the anhydrous crystal form C. The XRPD comparison of the wet sample and dried sample of the preparation method is shown in FIG. 15.
Method 2: about 199.63 mg of the compound of Formula (1) was weighed and suspended in 5.0 mL of a mixed solvent of ethyl acetate and isopropanol (with a volume ratio of 1:1) to obtain a suspension, and the suspension was magnetically stirred at 4-8° C. for 3 d to obtain a crystalline wet solid, which was the anhydrous crystal form C. The anhydrous crystal form C was air-dried at room temperature for 1 d and converted into the anhydrous crystal form A. The XRPD comparison of the wet sample and dried sample of the preparation method is shown in FIG. 19.

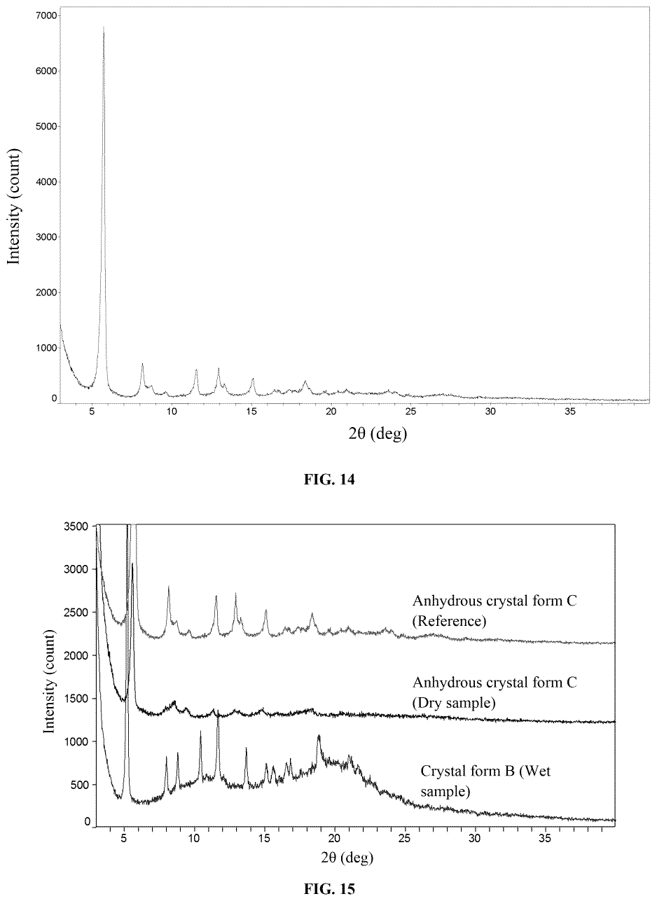

The anhydrous crystal form C has an XRPD pattern substantially as shown in FIG. 14.

The XRPD pattern of the anhydrous crystal form C has characteristic diffraction peaks at 2θ angles listed in Table 3.

TABLE 3

| 2θ | d-spacing | Relative intensity |
|---|---|---|
| (±0.2°) | (Å) | (%) |
| 5.73 | 15.42 | 100.00 |
| 8.16 | 10.82 | 8.90 |
| 8.73 | 10.12 | 2.60 |
| 9.59 | 9.21 | 1.10 |
| 11.55 | 7.65 | 7.10 |
| 12.94 | 6.84 | 7.50 |
| 13.29 | 6.66 | 3.10 |
| 15.10 | 5.86 | 4.60 |
| 16.44 | 5.39 | 1.30 |
| 16.66 | 5.32 | 1.20 |
| 17.38 | 5.10 | 0.80 |
| 18.37 | 4.83 | 3.40 |
| 19.67 | 4.51 | 1.20 |
| 20.43 | 4.34 | 1.10 |
| 20.96 | 4.23 | 1.40 |
| 23.61 | 3.77 | 1.50 |
| 24.02 | 3.70 | 1.10 |

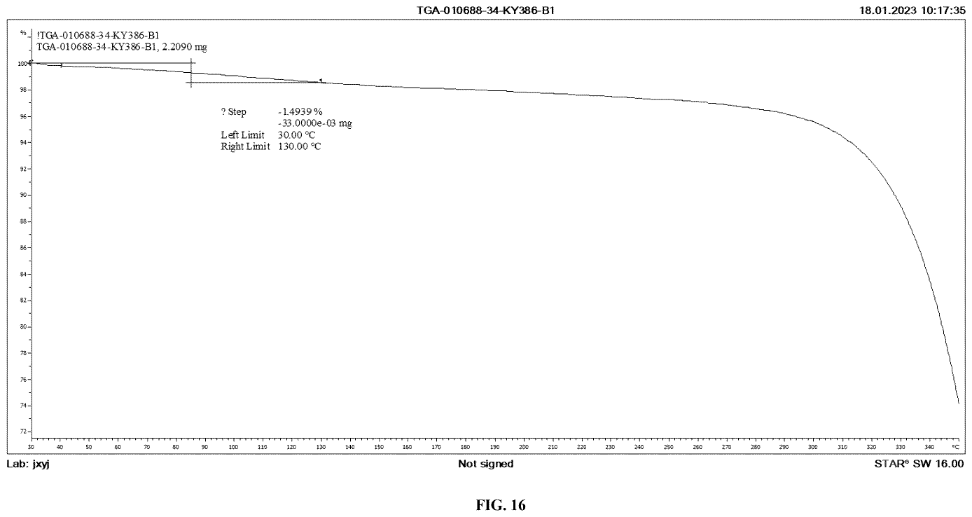

As shown in FIG. 16, a thermogravimetric analysis (TGA) curve of the anhydrous crystal form C indicates a weight loss of about 1.49% when a temperature is raised to about 130° C.

As shown in FIG. 17, a differential scanning calorimetry (DSC) curve of the anhydrous crystal form C exhibits an endothermic peak at about 162.13° C.

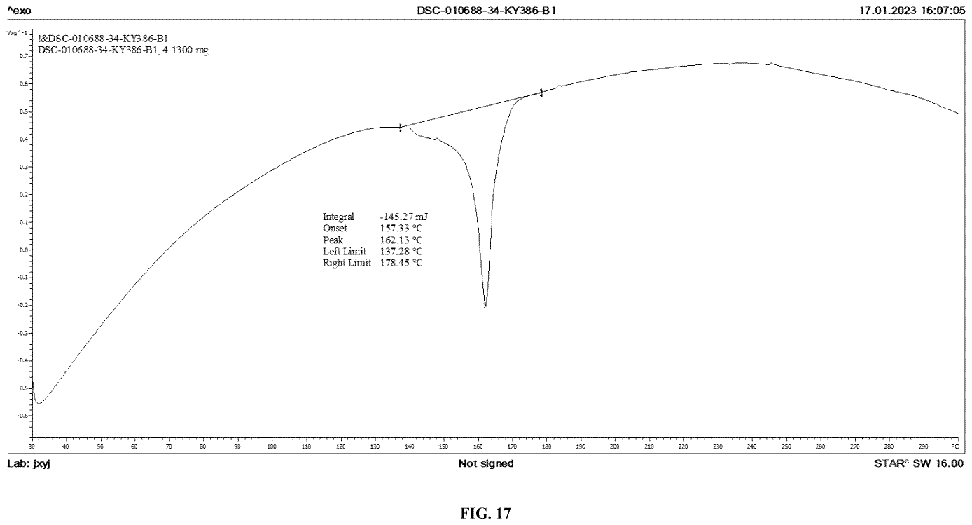

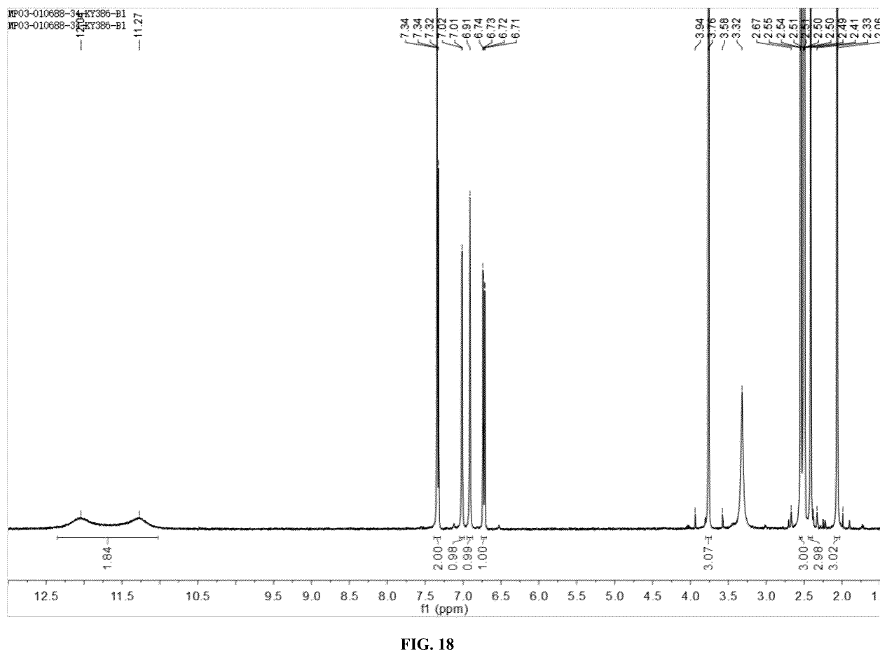

As shown in FIG. 18, the ¹H NMR spectrum of the anhydrous crystal form C indicates no significant solvent residue.

### Preparation Method for Crystal Form D of Compound of Formula (1)

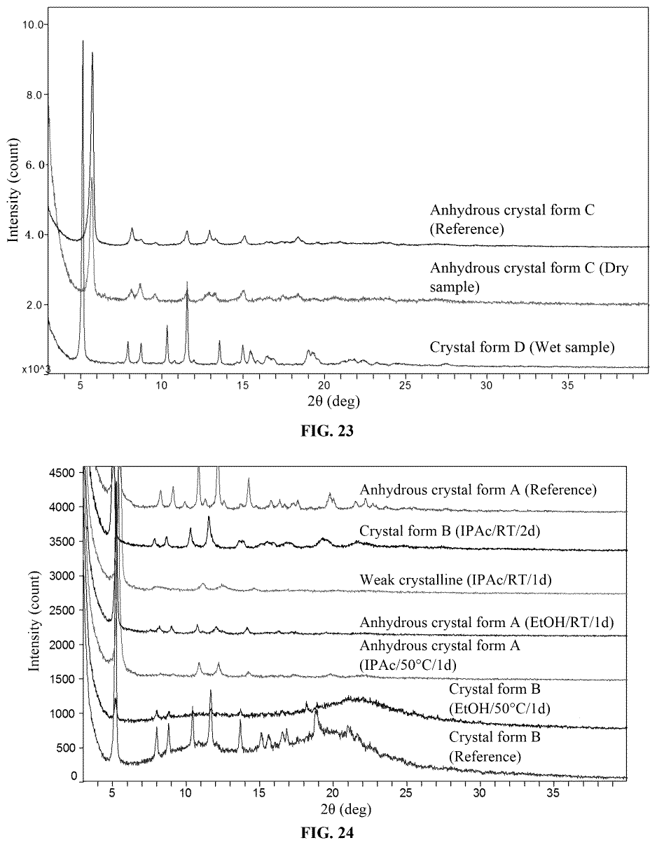

About 19.82 mg of the compound of Formula (1) was weighed and suspended in 5.0 mL of a mixed solvent of toluene and ethanol (with a volume ratio of 1:1) to obtain a suspension, and the suspension was magnetically stirred at 4-8° C. for 18 d to obtain a crystalline wet solid, which was the crystal form D. crystal form D was a metastable form. crystal form D was air-dried at room temperature for 1 d and converted into the anhydrous crystal form C. The XRPD comparison of the wet sample and dried sample of the preparation method is shown in FIG. 23.

crystal form D has an XRPD pattern substantially as shown in FIG. 22.

The XRPD pattern of crystal form D has characteristic diffraction peaks at 2θ angles listed in Table 4.

TABLE 4

| 2θ | d-spacing | Relative intensity |
|---|---|---|
| (±0.2°) | (Å) | (%) |
| 5.14 | 17.19 | 100.00 |
| 7.91 | 11.17 | 6.80 |
| 8.72 | 10.13 | 6.10 |
| 10.32 | 8.56 | 12.00 |
| 10.78 | 8.20 | 0.90 |
| 11.55 | 7.65 | 25.80 |
| 11.96 | 7.39 | 1.30 |
| 13.54 | 6.53 | 7.50 |
| 14.97 | 5.91 | 5.70 |
| 15.45 | 5.73 | 4.30 |
| 15.88 | 5.58 | 1.00 |
| 16.52 | 5.36 | 2.70 |
| 19.01 | 4.66 | 4.50 |
| 19.35 | 4.58 | 3.60 |
| 21.27 | 4.17 | 0.90 |
| 21.84 | 4.07 | 1.90 |
| 22.42 | 3.96 | 1.20 |
| 23.26 | 3.82 | 0.90 |
| 24.31 | 3.66 | 0.60 |
| 27.55 | 3.24 | 0.80 |

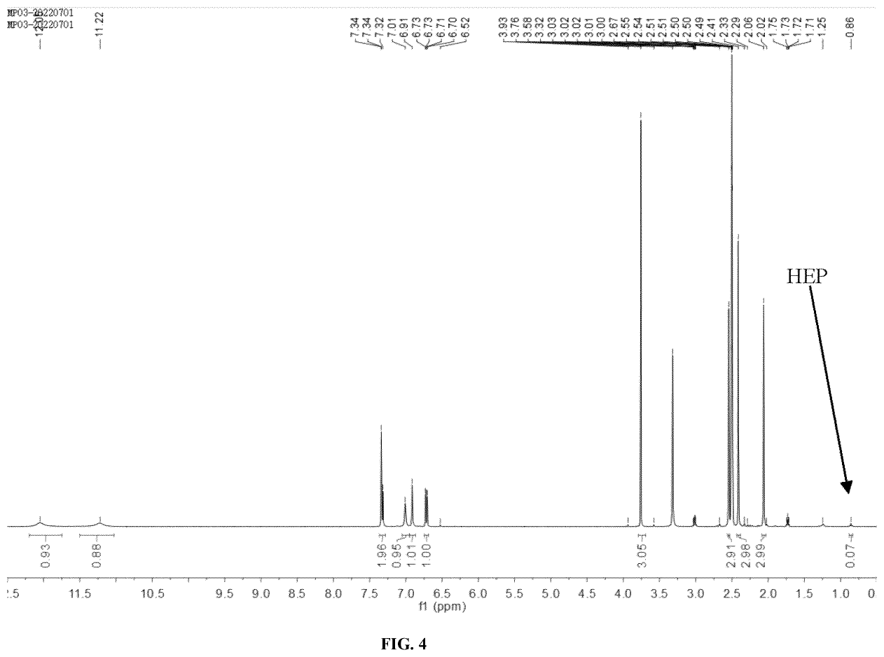

The XRPD comparison diagram of anhydrous crystal form A, crystal form B, anhydrous crystal form C, and crystal form D of Compound (1) is shown in FIG. 5.

### Example 3: Suspension Competition Experiment

In order to confirm a mutual conversion relationship between the anhydrous crystal form A and the anhydrous crystal form C, suspension competition experiments were conducted in EtOH (at room temperature (RT) and 50° C.) and IPAc (at room temperature and 50° C.). First, a suspension of Compound (1) equilibrated for 2 h at the corresponding temperature and solvent was filtered to obtain a saturated solution of Compound (1) at different temperatures and solvents, and solid mixtures of the anhydrous crystal form A and the anhydrous crystal form C were then added.

As shown in Table 5 and FIG. 24, a mixture of the anhydrous crystal form A and the anhydrous crystal form C was converted into the anhydrous crystal form A and crystal form B, indicating that crystal form B is relatively more stable in the case of the wet sample within a temperature range from room temperature to 50° C. The anhydrous crystal form A is a more thermodynamically stable anhydrous form from room temperature to 50° C.

TABLE 5

| | | Temperature | Competition for 1 d | Competition for 2 d |
|---|---|---|---|---|
| Starting crystal form | Solvent | (° C.) | (XPRD) | (XPRD) |
| Mixture of | EtOH | 50 | crystal form B | / |
| anhydrous crystal | IPAc | | Anhydrous crystal | / |
| form A and | | | form A | |
| anhydrous crystal | EtOH | RT | Anhydrous crystal | / |
| form C | | | form A | |
| | IPAc | | Weekly crystal | crystal form B |
| | | | form | |

/: Not detected.

### Example 4: Solid-Form Stability

The anhydrous crystal form A and the anhydrous crystal form C were each stored under long-term (25° C./60% RH) and accelerated (40° C./75% RH) conditions for 10 d, and the HPLC purity and changes in crystal form were then tested.

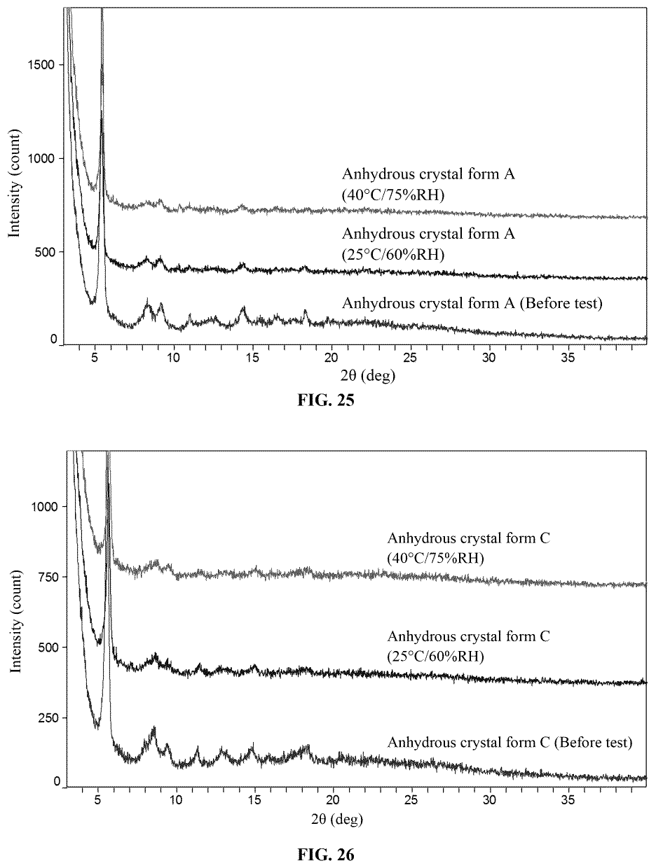

As shown in Table 6 and FIGS. 25-26, neither changes in crystal form nor significant decrease in HPLC purity was observed for the anhydrous crystal form A and the anhydrous crystal form C, indicating that both the anhydrous crystal form A and the anhydrous crystal form C exhibit good physical and chemical stability under the test conditions.

TABLE 6

| | | 25°C/ | | 40°C/ | |
|---|---|---|---|---|---|
| | | 60% RH, 10 d | | 75% RH, 10 d | |
| | Initial | Crystal | | Crystal | |
| | purity | form | Purity | form | Purity |
| Solid form | (Area %) | Change | (Area %) | Change | (Area %) |
| Anhydrous | 99.55 | No | 99.67 | No | 99.60 |
| crystal form A | | | | | |
| Anhydrous | 99.84 | No | 99.87 | No | 99.90 |
| crystal form C | | | | | |

### Example 5: High-Temperature Stability

The anhydrous crystal form A and the anhydrous crystal form C were each stored at 60° C. for 10 d, and the HPLC purity and changes in crystal form were then tested.

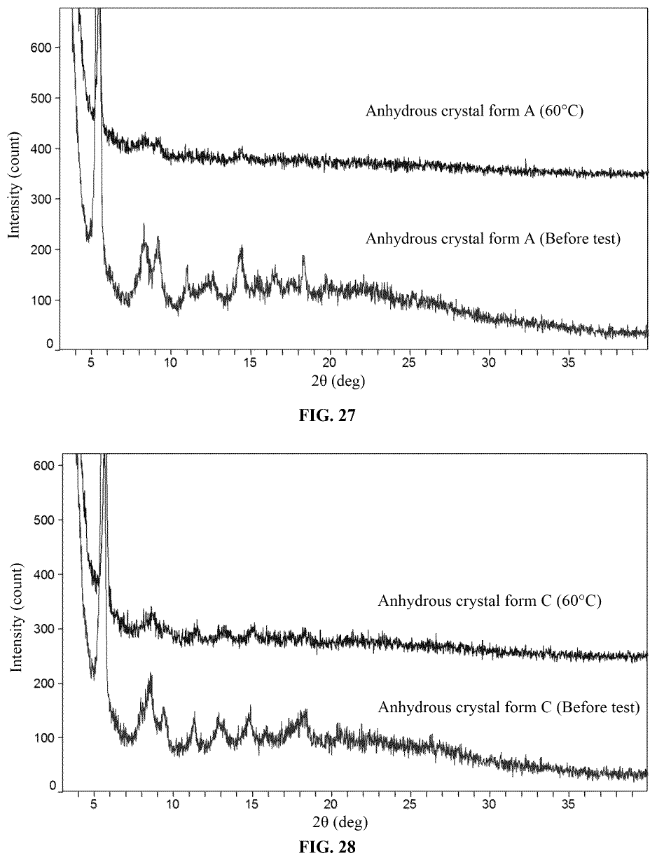

As shown in Table 7 and FIGS. 27-28, neither changes in crystal form nor significant decrease in HPLC purity was observed for the anhydrous crystal form A and the anhydrous crystal form C, indicating that both the anhydrous crystal form A and the anhydrous crystal form C exhibit good physical and chemical stability under the test conditions.

TABLE 7

| | | 60° C., 10 d | |
|---|---|---|---|
| | | Crystal form | Purity |
| Solid form | Initial purity | Change | (Area %) |
| Anhydrous crystal form A | 99.55 | No | 99.63 |
| Anhydrous crystal form C | 99.84 | No | 99.86 |

### Example 6: Hygroscopicity Evaluation

To evaluate a stability risk of the sample at 25° C. with varying humidity, the anhydrous crystal form A and the anhydrous crystal form C were subjected to the DVS test, and solid samples after DVS test were collected for XRPD test.

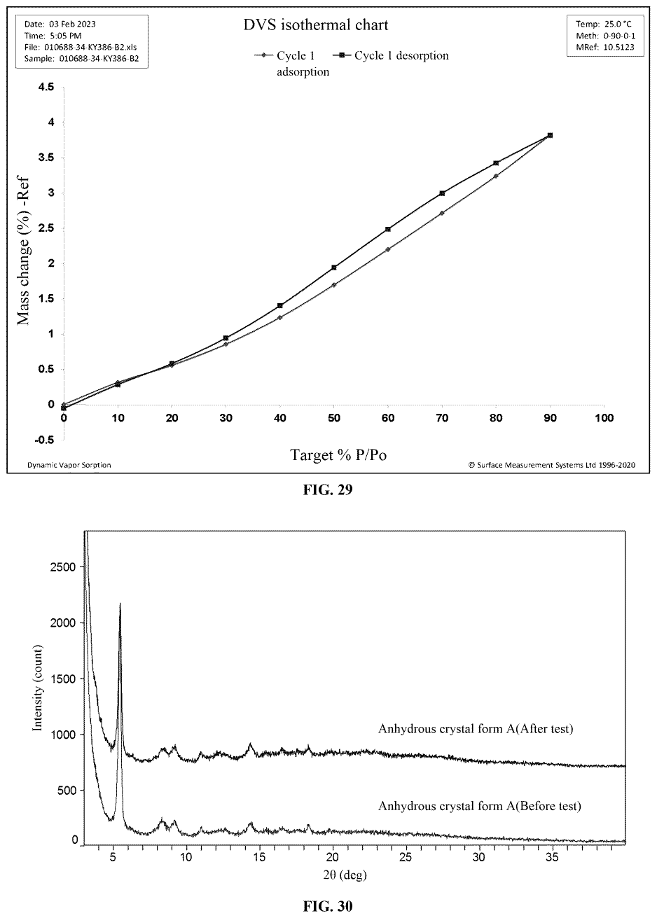

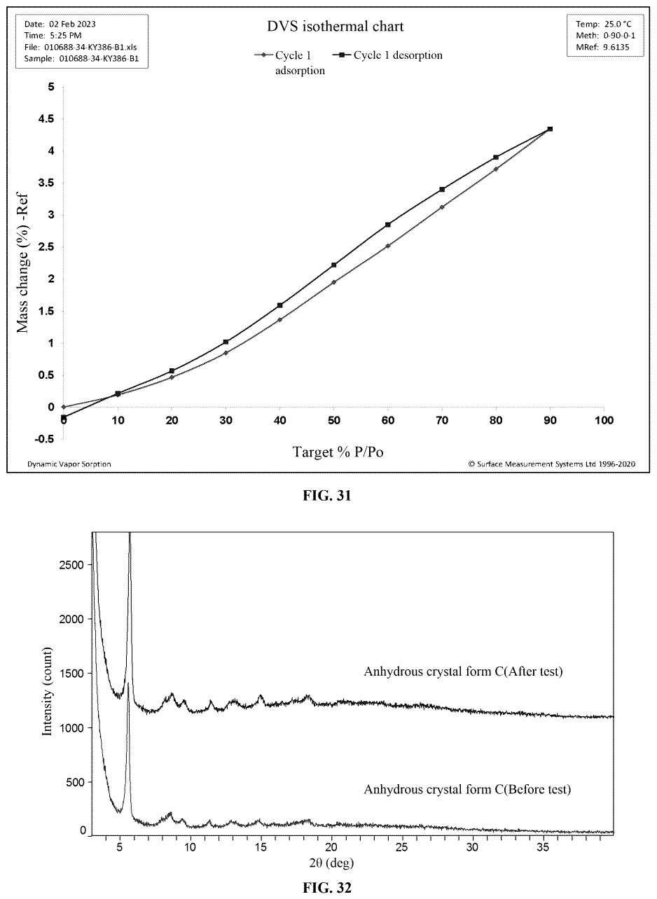

As shown in Table 8, the anhydrous crystal form A absorbs 3.24% of water under 80% RH conditions, indicating that the sample is of slight hygroscopicity. The anhydrous crystal form C absorbs water and shows a weight gain by 3.71%, indicating that the sample is also of slight hygroscopicity. Results of the DVS and XRPD test are shown in FIGS. 29-32. Before and after the DVS test, no changes in crystal form were observed for the anhydrous crystal form A and the anhydrous crystal form C.

TABLE 8

| | Sample weight | Whether crystal form | |
|---|---|---|---|
| | change at 80% RH | changes before and | |
| Solid form | (%) | after test | Hygroscopicity * |
| Anhydrous | 3.24 | No | Slightly |
| crystal form A | | | hygroscopicity * |
| Anhydrous | 3.71 | No | Slight |
| crystal form C | | | hygroscopicity |

* Hygroscopicity is based on the weight gain of the sample when the humidity rises to 80% RH at 25° C. Chinese Pharmacopoeia (2020 Edition), a weight gain of 2%-15% indicates hygroscopicity.

### Example 7: Polymorph Screening Experiment

### Slow Evaporation

About 20 mg of the compound of Formula (1) was weighed and dissolved in 1.0-3.0 mL of solvent. When the Compound was not completely dissolved, a suspension was filtered through a polytetrafluoroethylene to obtain a filtrate, and the filtrate was used in subsequent steps. A vial containing the clear solution was sealed with a perforated sealing film and allowed to evaporate slowly at room temperature. The obtained solid was collected and subjected to XRPD test. Test results are shown in Table 9, the anhydrous crystal form A is obtained.

TABLE 9

| | Solvent | |
|---|---|---|
| Solvent (v:v) | volume (mL) | Solid crystal form |
| Acetone/ACN (3:1) | 1.8 | Anhydrous crystal form A |
| EtOAc | 2.5 | Anhydrous crystal form A |
| THF/H₂O (1:1) | 2.4 | Anhydrous crystal form A |
| CH₂Cl₂/CPME (1:1) | 2.4 | Anhydrous crystal form A |
| Toluene/THF (2:1) | 1.8 | Anhydrous crystal form A |

### Slurry Conversion at Low Temperature (4-8° C.)

About 20 mg of the compound of Formula (1) was weighed and suspended in 0.5 mL of solvent to obtain a suspension. The suspension was magnetically stirred at 4-8° C. for about 18 d, and a solid was then isolated and subjected to XRPD test. Test results are shown in Table 10, the anhydrous crystal form A/C is obtained.

TABLE 10

| Solvent (v:v) | Solid crystal form |
|---|---|
| MeOH | Anhydrous crystal form A |
| Acetone/H₂O (1:2) | Anhydrous crystal form A |
| EtOAc/IPA (1:1) | Anhydrous crystal form A |
| MTBE/MEK (1:1) | Anhydrous crystal form A |
| CH₂Cl₂/ACN (1:3) | Anhydrous crystal form C |
| 2-MeTHF/HEP (1:1) | Anhydrous crystal form A |
| THF/IPAc (1:4) | Anhydrous crystal form A |
| CPME/THF (4:1) | Anhydrous crystal form A |

### Slurry Conversion at Room Temperature

About 20 mg of the compound of Formula (1) was weighed and suspended in 0.5 mL of solvent to obtain a suspension. The suspension was magnetically stirred at room temperature for about 14-18 d, and a solid was then isolated and subjected to XRPD test. Test results are shown in Table 11, the anhydrous crystal form A/C or crystal form B is obtained.

TABLE 11

| Solvent (v:v) | Solid crystal form |
|---|---|
| EtOH | Anhydrous crystal form A |
| ACN/H₂O (1:1) | Anhydrous crystal form C |
| IPAc | Anhydrous crystal form A |
| CH₂Cl₂/MeOH (1:4) | Anhydrous crystal form A |
| MEK/MCH (1:2) | Anhydrous crystal form A |
| 1,4-Dioxane/Toluene (1:3) | Anhydrous crystal form A |
| IPA/H₂O (1:1) | Anhydrous crystal form A |
| 2-MeTHF/MTBE (1:2) | crystal form B |
| MIBK/ACN (1:1) | Anhydrous crystal form A |
| Acetone/EtOAc (1:5) | Anhydrous crystal form A |

### Slurry Conversion at 50° C.

About 20 mg of the compound of Formula (1) was weighed and suspended in 0.5 mL of solvent to obtain a suspension. The suspension was magnetically stirred at 50° C. for about 7 d, and then stirred at 5° C. for about 11 d, and a solid was then isolated and subjected to XRPD test. Test results are shown in Table 12, the anhydrous crystal form A or crystal form B is obtained.

TABLE 12

| Solvent (v:v) | Solid crystal form |
|---|---|
| IPA | crystal form B |
| MEK/Toluene (1:2) | crystal form B |
| CPME/EtOH (1:1) | Anhydrous crystal form A |
| THF/H₂O (1:6) | Anhydrous crystal form A |
| 1,4-Dioxane/n-Hexane (1:3) | Anhydrous crystal form A |

### Cooling Crystallization

About 20 mg of the compound of Formula (1) was weighed and suspended in 1.0 mL of solvent to obtain a suspension, the suspension was then heated to 50° C. and equilibrated for about 2 h, and filtered through polytetrafluoroethylene to obtain a filtrate, the filtrate was then slowly cooled to room temperature in a water bath, and a solid was then isolated and subjected to XRPD test. Test results are shown in Table 13, the anhydrous crystal form A/C is obtained.

TABLE 13

| Solvent (v:v) | Solid crystal form |
|---|---|
| MeOH | Anhydrous crystal form A |
| ACN/H₂O (3:1) | Anhydrous crystal form C |

### Gas-Liquid Diffusion

About 20 mg of the compound of Formula (1) was weighted and dissolved in 0.2-2.0 mL of a good solvent and filtered to obtain a clear solution; a vial containing the clear solution was placed into a 20 mL vial containing 3 mL of a volatile anti-solvent; the 20 mL vial was sealed with a cap and kept at room temperature to allow sufficient time for organic vapor to interact with the solution; and a solid was then isolated and subjected to XRPD test. Test results are shown in Table 14, the anhydrous crystal form A is obtained.

TABLE 14

| | Good solvent | | |
|---|---|---|---|
| Good solvent (v:v) | volume (mL) | Anti-solvent | Solid crystal form |
| 1,4-Dioxane/ | 0.6 | HEP | Anhydrous crystal form |
| Toluene (4:1) | | | A |
| MEK/ACN (4:1) | 1.2 | n-Pentane | Anhydrous crystal form |
| | | | A |
| CHCl₃/MIBK (2:1) | 2.0 | n-Pentane | Anhydrous crystal form |
| | | | A |

### Gas-Solid Diffusion

About 20 mg of the compound of Formula (1) was weighed and placed into a 3 mL vial, which was then placed into a 20 mL vial containing 3 mL of a volatile solvent, the 20 mL vial was sealed with a cap and stored at room temperature for 18 d to allow solvent vapor to interact with the sample, and a solid was collected and subjected to XRPD test. Test results are shown in Table 15, the anhydrous crystal form A or crystal form B is obtained.

TABLE 15

| Solvent | Solid crystal form |
|---|---|
| Acetone | Anhydrous crystal form A |
| EtOH | Anhydrous crystal form A |
| EtOAc | Anhydrous crystal form A |
| THF | Anhydrous crystal form A |
| DMSO | crystal form B |

### Water Vapor Stress

About 20 mg of the compound of Formula (1) was weighed and placed in a 3 mL vial, which was exposed to conditions of RT-75% relative humidity, RT-85% relative humidity, and RT-97% relative humidity for 18 d, and a solid was collected and subjected to XRPD test. Test results are shown in Table 16, only the anhydrous crystal form A is obtained.

TABLE 16

| Temperature and humidity | |
|---|---|
| (° C.-RH %) | Solid crystal form |
| RT-75% RH | Anhydrous crystal form A |
| RT-85% RH | Anhydrous crystal form A |
| RT-97% RH | Anhydrous crystal form A |

### Polymer-Induced Crystallization

About 20 mg of the compound of Formula (1) was weighed and dissolved in 0.8-3.0 mL of solvent. When the Compound was not completely dissolved, a suspension was filtered through a polytetrafluoroethylene to obtain a filtrate, and the filtrate was used in subsequent steps. About 1-2 mg of polymer was added to each vial, each vial was sealed with sealing film, and pinholes were pierced to allow for slow evaporation at room temperature; and the obtained solid was collected and subjected to XRPD test. Test results are shown in Table 17, the anhydrous crystal form A, crystal form B, the anhydrous crystal form A+crystal form B are obtained.

TABLE 17

| | | Solvent | |
|---|---|---|---|
| | | volume | |
| Solvent (v:v) | Polymer | (mL) | Solid crystal form |
| EtOH/CHCl₃ | Copovidone | 2.0 | Anhydrous crystal |
| (2:1) | | | form A |
| MEK/H₂O (5:1) | Polyethylene glycol | 2.4 | crystal form B |
| 1,4-Dioxane | Ethyl cellulose | 0.8 | Anhydrous crystal |
| | | | form A + crystal |
| | | | form B |
| THF/HEP (1:1) | Polyvinylpyrrolidone | 3.0 | Anhydrous crystal |
| | | | form A |

### Grinding

About 20 mg of the compound of Formula (1) was ground in a mortar (or ground using a solvent), and a solid was then isolated and subjected to XRPD test. Test results are shown in Table 18, the anhydrous crystal form A is obtained.

TABLE 18

| Solvent | Solvent volume (mL) | Solid crystal form |
|---|---|---|
| H₂O | 0.02 × 3 | Anhydrous crystal form A |
| EtOH | 0.02 × 3 | Anhydrous crystal form A |
| Acetone | 0.02 × 3 | Anhydrous crystal form A |

### Temperature Cycling

About 20 mg of the compound of Formula (1) was weighed and suspended in 0.5 mL of solvent to obtain a suspension, the suspension was magnetically stirred under the following temperature cycling conditions, and a solid was then isolated and subjected to XRPD test. Test results are shown in Table 19, the anhydrous crystal form A, crystal form B, and the anhydrous crystal form C are obtained.

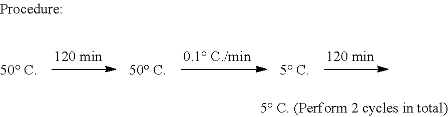

TABLE 19

| Solvent (v:v) | Solid crystal form |
|---|---|
| Acetone/IPA (1:5) | Crystal form B |
| IPAc/CPME (1:1) | Anhydrous crystal form A |
| ACN | Anhydrous crystal form C |
| MeOH/MEK (4:1) | Anhydrous crystal form A |
| EtOAc/CHCl₃ (5:1) | Anhydrous crystal form A |
| THF/HEP (1:5) | Anhydrous crystal form A |
| 1,4-Dioxane /MTBE (1:3) | Anhydrous crystal form A |
| MIBK/MCH (1:1) | Anhydrous crystal form A |

### Rotary Evaporation

About 50 mg of the compound of Formula (1) was weighted and dissolved in 0.5-1.4 mL of a solvent and filtered to obtain a clear solution; the solution was subjected to rotary evaporation at 50° C., and a solid was then isolated and subjected to XRPD test. Test results are shown in Table 20, the anhydrous crystal form A is obtained.

TABLE 20

| Solvent | Solvent volume (mL) | Solid crystal form |
|---|---|---|
| CH₂Cl₂ | 1.4 | Anhydrous crystal form A |
| CH₂Cl₂ | 80.0 | Anhydrous crystal form A* |

*3.0 g of the compound of Formula (1).

What is claimed is:

1. An anhydrous crystal form A of a compound of Formula (1), 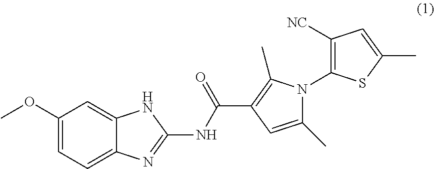
wherein an XRPD pattern of the anhydrous crystal form A has characteristic diffraction peaks at 2θ angles of 5.36±0.2°, 12.17±0.2°, and 10.85±0.2°;
preferably, the XRPD pattern of the anhydrous crystal form A further has characteristic diffraction peaks at one or more of the following 2θ angles of 14.26±0.2°, 9.12±0.2°, and 8.26±0.2°;
preferably, the XRPD pattern of the anhydrous crystal form A further has characteristic diffraction peaks at one or more of the following 2θ angles of 19.77±0.2°, 20.03±0.2°, and 22.17±0.2°; and
preferably, the anhydrous crystal form A has an XRPD pattern substantially as shown in FIG. 7.

2. An anhydrous crystal form C of a compound of Formula (1), 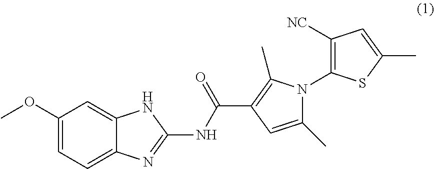
wherein an XRPD pattern of the anhydrous crystal form C has characteristic diffraction peaks at 2θ angles of 5.73±0.2°, 8.16±0.2°, and 12.94±0.2°;
preferably, the XRPD pattern of the anhydrous crystal form C further has characteristic diffraction peaks at one or more of the following 2θ angles of 11.55±0.2°, 15.10±0.2°, and 18.37±0.2°;
preferably, the XRPD pattern of the anhydrous crystal form C further has characteristic diffraction peaks at one or more of the following 2θ angles of 13.29±0.2°, 8.73±0.2°, and 23.61±0.2°; and
preferably, the anhydrous crystal form C has an XRPD pattern substantially as shown in FIG. 14.

3. A pharmaceutical composition, comprising the anhydrous crystal form A according to claim 1, and one or more pharmaceutically acceptable carriers.

4. A method for preventing and/or treating a disease or condition mediated by DHX33, comprising administering the anhydrous crystal form A according to claim 1 or a pharmaceutical composition comprising the anhydrous crystal form A and one or more pharmaceutically acceptable carriers to a subject; wherein preferably, the disease is selected from cancer, viral infection, and inflammation mediated by DHX33.

5. A preparation method for the anhydrous crystal form A according to claim 1, comprising any-one of the following methods:
(1) suspending the compound of Formula (1) in a solvent and stirring at 5-35° C. for 1-3 d to obtain a crystal wet sample, wherein the crystal wet sample is a crystal form B; and drying the crystal form B at room temperature to convert the crystal form B into the anhydrous crystal form A; wherein the solvent is selected from ethanol or a mixed solvent of ethyl acetate and isopropanol;
(2) suspending the compound of Formula (1) in a solvent and stirring at 4-8° C. for 14-18 d, and isolating a resulting solid to obtain the anhydrous crystal form A, wherein the solvent is selected from methanol, a mixed solvent of acetone and water, a mixed solvent of ethyl acetate and isopropanol, a mixed solvent of methyl tert-butyl ether and methyl ethyl ketone, a mixed solvent of 2-methyltetrahydrofuran and n-heptane, a mixed solvent of tetrahydrofuran and isopropyl acetate, and a mixed solvent of cyclopentyl methyl ether and tetrahydrofuranyl; and
(3) suspending the compound of Formula (1) in a solvent and stirring at room temperature for 14-18 d, and then isolating a resulting solid to obtain the anhydrous crystal form A; wherein the solvent is selected from ethanol, isopropyl acetate, a mixed solvent of dichloromethane and methanol, a mixed solvent of methyl ethyl ketone and methylcyclohexane, a mixed solvent of 1,4-dioxane and toluene, a mixed solvent of isopropanol and water, a mixed solvent of methyl isobutyl ketone and acetonitrile, and a mixed solvent of acetone and ethyl acetate.

6. A preparation method for the anhydrous crystal form C according to claim 2, comprising one of the following methods:
(1) suspending the compound of Formula (1) in a solvent and stirring at 5-35° C. for 1-3 d to obtain a crystal wet sample, wherein the crystal wet sample is a crystal form B; and converting the crystal form B into the anhydrous crystal form C by drying at 50-80° C.; wherein the solvent is selected from ethanol or a mixed solvent of ethyl acetate and isopropanol;
(2) suspending the compound of Formula (1) in a solvent and stirring at 4-8° C. for 3 d, and isolating a resulting solid to obtain the anhydrous crystal form C; wherein the solvent is selected from a mixed solvent of ethyl acetate and isopropanol; and
(3) suspending the compound of Formula (1) in a solvent and stirring at room temperature for 14-18 d, and isolating a resulting solid to obtain the anhydrous crystal form C; wherein the solvent is selected from a mixed solvent of acetonitrile and water.

7. A pharmaceutical composition, comprising the anhydrous crystal form C according to claim 2 and one or more pharmaceutically acceptable carriers.

8. A method for preventing and/or treating a disease or condition mediated by DHX33, comprising administering the anhydrous crystal form C according to claim 2 or a pharmaceutical composition comprising the anhydrous crystal form C and one or more pharmaceutically acceptable carriers to a subject; wherein preferably, the disease is selected from cancer, viral infection, and inflammation mediated by DHX33.
